(19) United States
(12) Patent Application Publication
(10) Pub. No.: US 2021/0145798 A1
(43) Pub. Date: May 20, 2021

(54) METHOD FOR TREATING AMYOTROPHIC LATERAL SCLEROSIS AND METHOD FOR SUPPRESSING PROGRESS OF AMYOTROPHIC LATERAL SCLEROSIS

(71) Applicant: MITSUBISHI TANABE PHARMA CORPORATION, Osaka-shi (JP)

(72) Inventors: Charlotte MERRILL, Jersey City, NJ (US); Wendy AGNESE, Jersey City, NJ (US); Nazem ATASSI, Boston, MA (US); Tara GRABOWSKY, King Of Prussia, PA (US); Takeshi SAKATA, Osaka-shi (JP)

(73) Assignee: MITSUBISHI TANABE PHARMA CORPORATION, Osaka-shi (JP)

(21) Appl. No.: 16/753,669

(22) PCT Filed: Oct. 4, 2018
(86) PCT No.: PCT/US2018/054299

§ 371 Date: Apr. 3, 2020

Related U.S. Application Data
(63) Continuation-in-part of application No. PCT/US2018/020184, filed on Feb. 28, 2018.
(60) Provisional application No. 62/567,873, filed on Oct. 4, 2017.

(51) Int. Cl. A61K 31/4152; A61P 25/28

(52) U.S. Cl. CPC A61K 31/4152; A61P 25/28

(57) ABSTRACT

A method for treating amyotrophic lateral sclerosis includes administering an effective amount of 3-methyl-1-phenyl-2-pyrazolin-5-one or a physiologically acceptable salt thereof to a patient who is in need thereof and meets two or more features selected from a group of identified features.

### CROSS-REFERENCE TO RELATED APPLICATIONS

The present application is based upon and claims the benefit of priority to International Application No. PCT/US2018/020184, filed Feb. 28, 2018, which is based upon and claims the benefit of priority to U.S. Provisional Application No. 62/567,873, filed Oct. 4, 2017. The entire contents of these applications are incorporated herein by reference.

### BACKGROUND OF THE INVENTION

### Field of the Invention

The present invention relates to a method for treating amyotrophic lateral sclerosis (hereinafter also referred to as ALS) or suppressing progress of the disease, and a method for treating a symptom caused by ALS or suppressing progress of the symptom.

### Description of Background Art

As a medication effective for suppressing progress of ALS, there has been approved “riluzole” that suppresses glutamic acid transmission in glutamatergic nerve. However, it has also been reported that the effectiveness of riluzole cannot be confirmed. Thus, the evaluation of this drug is not consistent.

ALS, which is one type of motor neuron disease, is an intractable disease. ALS starts with initial symptoms such as weakness in hands, movement disorders with fingers and fascicular contraction in upper limbs. Thereafter, ALS has amyotrophia and/or muscular weakness, bulbar paralysis and fascicular contraction in muscles, and it finally leads to respiratory failure. ALS is divided into upper limb, bulbar, lower limb and mixed types, depending on a site of onset. In all of these types, as symptoms progress, a systemic muscle group is affected.

### SUMMARY OF THE INVENTION

A method for treating amyotrophic lateral sclerosis at an early stage according to an embodiment of the present invention includes administering an effective amount of 3-methyl-1-phenyl-2-pyrazolin-5-one or a physiologically acceptable salt thereof to a patient who has at least two Features of identified Feature 1 to Feature 55. The identified Feature 1 to Feature 55 are selected from the following.

1. Abnormality of gait
2. Aldolase test
3. Antinuclear antibodies (ANA) test
4. Cervical spondylosis without myelopathy
5. Creatine kinase (CK):(CPK) test
6. Cyanocobalamin (Vitamin B-12) test
7. Degeneration of cervical intervertebral disc
8. Displacement of cervical intervertebral disc without myelopathy

### 9. Dysphagia

10. Folic acid; serum test
11. Serum immunofixation electrophoresis test
12. Magnetic resonance imaging test
13. Manual therapy techniques
14. Muscle weakness
15. Needle electromyography
16. Acquired deformities of ankle and foot
17. Malaise and fatigue
18. Physical therapy evaluation
19. Serum protein electrophoretic fractionation and quantitation test
20. Erythrocyte sedimentation rate test
21. Spinal stenosis in cervical region
22. Swallowing function test
23. Therapeutic procedure for neuromuscular reeducation
24. Therapeutic procedure for therapeutic exercises
25. Thyroid stimulating hormone (TSH) test
26. Unspecified hereditary and idiopathic peripheral neuropathy
27. Nervous system disorders
28. Hereditary and degenerative nervous system conditions
29. Connective tissue disease
30. Non-traumatic joint disorders
31. Multiple sclerosis

### 32. Paraplegia

### 33. Paralysis

34. Other diagnostic nervous system procedures
35. Durable Medical Equipment (DME) and supplies
36. Physical therapy

### 37. Laryngoscopy

38. Spinal puncture
39. Treatment of speech

### 40. Riluzole

### 41. Baclofen

### 42. Pyridostigmine

### 43. Anticonvulsants

### 44. Diazepam

### 45. Hydrocodone

### 46. Propoxyphene

### 47. Sympathomimetic Agents

### 48. Glycopyrrolate

### 49. Prednisone

### 50. Pregabalin

### 51. Clonazepam

### 52. Tizanidine

### 53. Levodopa or Carbidopa

### 54. Quinine

### 55. Tolterodine

A method for suppressing progress of amyotrophic lateral sclerosis at an early stage according to another embodiment of the present invention includes administering an effective amount of 3-methyl-1-phenyl-2-pyrazolin-5-one or a physiologically acceptable salt thereof to a patient who has at least two Features of identified feature 1 to feature 55. The identified Feature 1 to Feature 55 are selected from the following.

1. Abnormality of gait
2. Aldolase test
3. Antinuclear antibodies (ANA) test
4. Cervical spondylosis without myelopathy
5. Creatine kinase (CK):(CPK) test
6. Cyanocobalamin (Vitamin B-12) test
7. Degeneration of cervical intervertebral disc
8. Displacement of cervical intervertebral disc without myelopathy

### 9. Dysphagia

10. Folic acid; serum test
11. Serum immunofixation electrophoresis test
12. Magnetic resonance imaging test
13. Manual therapy techniques
14. Muscle weakness
15. Needle electromyography
16. Acquired deformities of ankle and foot
17. Malaise and fatigue
18. Physical therapy evaluation
19. Serum protein electrophoretic fractionation and quantitation test
20. Erythrocyte sedimentation rate test
21. Spinal stenosis in cervical region
22. Swallowing function test
23. Therapeutic procedure for neuromuscular reeducation
24. Therapeutic procedure for therapeutic exercises
25. Thyroid stimulating hormone (TSH) test
26. Unspecified hereditary and idiopathic peripheral neuropathy
27. Nervous system disorders
28. Hereditary and degenerative nervous system conditions
29. Connective tissue disease
30. Non-traumatic joint disorders
31. Multiple sclerosis

### 32. Paraplegia

### 33. Paralysis

34. Other diagnostic nervous system procedures
35. Durable Medical Equipment (DME) and supplies
36. Physical therapy

### 37. Laryngoscopy

38. Spinal puncture
39. Treatment of speech

### 40. Riluzole

### 41. Baclofen

### 42. Pyridostigmine

### 43. Anticonvulsants

### 44. Diazepam

### 45. Hydrocodone

### 46. Propoxyphene

### 47. Sympathomimetic Agents

### 48. Glycopyrrolate

### 49. Prednisone

### 50. Pregabalin

### 51. Clonazepam

### 52. Tizanidine

### 53. Levodopa or Carbidopa

### 54. Quinine

### 55. Tolterodine

A method for suppressing progress of amyotrophic lateral sclerosis at an early stage according to yet another embodiment of the present invention includes administering an effective amount of 3-methyl-1-phenyl-2-pyrazolin-5-one or a physiologically acceptable salt thereof to a patient who has at least two Features of identified Feature 1 to Feature 11. The identified Feature 1 to Feature 11 are selected from the following.

1. Malaise and fatigue, or Muscle weakness
2. Non-traumatic joint disorder or Acquired deformities of ankle and foot
3. Connective tissue disease
4. Skin disorder
5. Nervous system disorder
6. Any change in speech
7. Office visit to: Physical therapy, Neurologist, Orthopedic surgeon, Gastroenterologist, or Otolaryngologist
8. Magnetic resonance imaging test, or Needle electromyography

### 9. Riluzole, Baclofen, Pyridostigmine, Anticonvulsants

10. Unusual increase in healthcare resource utilization
11. Creatine kinase (CK):(CPK) test, Cyanocobalamin (Vitamin B-12) test, or Antinuclear antibodies (ANA) test.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 illustrates top 20 two-Feature combinations according to an embodiment of the present invention based on mutual information rank and values in periods of three to six months prior to patients are diagnosed as having ALS;

FIG. 2 illustrates top 20 two-Feature combinations according to an embodiment of the present invention based on mutual information rank and values in periods of six to nine months prior to patients are diagnosed as having ALS;

FIG. 3 illustrates top 20 two-Feature combinations according to an embodiment of the present invention based on mutual information rank and values in periods of nine to twelve months prior to patients are diagnosed as having ALS;

FIG. 4 illustrates top 20 two-Feature combinations according to an embodiment of the present invention based on mutual information rank and values in periods of twelve to eighteen months prior to patients are diagnosed as having ALS;

FIG. 5 illustrates selected 3 Feature combinations according to an embodiment of the present invention by mutual information rank;

FIG. 6 illustrates selected 4 Feature combinations according to an embodiment of the present invention by mutual information rank;

FIG. 7 illustrates selected 5 Feature combinations according to an embodiment of the present invention by mutual information rank

FIG. 8 illustrates score distributions of Targets (ALS patients) and Controls (control patients) of the model;

FIG. 9 illustrates ROC (Receiver Operating Characteristic) curve; True Positive Rate, False Positive Rate, and PPV vs. Threshold of the model;

FIG. 10 illustrates a confusion matrix when the probability threshold is set to 0.1; and

FIG. 11 illustrates a confusion matrix when the probability threshold is set to 0.9.

### DETAILED DESCRIPTION OF THE EMBODIMENTS

Embodiments will now be described with reference to the accompanying drawings, wherein like reference numerals designate corresponding or identical elements throughout the various drawings.

Causal factors of ALS have not yet been sufficiently elucidated. The following hypotheses have been proposed as main causal factors of ALS: (1) autoimmune theory (appearance of an autoantibody against a Ca channel); (2) excessive excitatory amino acid and/or toxication theory (an increase in extracellular glutamic acid and transport disorders of glutamic acid); (3) oxidative stress disorder theory (Cu/Zn superoxide dismutase (SOD) genetic abnormality and nerve cell damage caused by free radicals); (4) cytoskeleton disorder theory (accumulation of neurofilament in motor nerve cells and appearance of inclusion bodies); and (5) deficiency of neurotrophic factors.

Examples of symptoms caused by ALS include clinical symptoms such as decreased respiratory function, speech language impairment, swallowing disorder, movement disorder of limbs, and the like. In the present invention, a decrease in respiratory function is a preferable example. This term should be interpreted in the broadest sense as long as it conforms to the above definition and should not be construed in a way that is confined to differences in disease names. Whether or not it is a disease equivalent to ALS can be diagnosed by a doctor.

Further, a preferable example of treating and/or suppressing progress of ALS or a symptom caused by ALS is suppression of a decrease in respiratory function in amyotrophic lateral sclerosis.

An active ingredient of the drug of the present invention is 3-methyl-1-phenyl-2-pyrazolin-5-one. 3-methyl-1-phenyl-2-pyrazolin-5-one can be represented by the following structural formula. 3-methyl-1-phenyl-2-pyrazolin-5-one has tautomers represented by the following structural formula. However, as the active ingredient of the drug of the present invention, any of these isomers may be used.

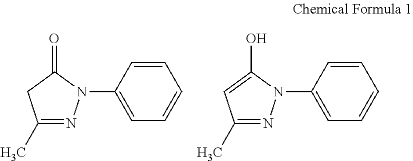

When any disease is found in a human body, appropriate treatment may be performed by a doctor. Drug treatment is also one of treatments. However, in drug treatment, it may be necessary to continue to administer drugs until the disease is cured. In contrast, for the drug and method of an embodiment of the present invention, during a drug treatment period, two or more 14-day drug holiday periods are provided, that is, a unit period including an administration period and a drug holiday period is repeated two or more times. Here, when an administration period and a drug holiday period are repeated two or more times, an end of this period is definitely a drug holiday period. However, it is not necessary to provide the last drug holiday period. That is, for example, when an administration period and a drug holiday period are repeated twice, this is a case of “an administration period, a drug holiday period, an administration period, and a drug holiday period. However, a case of “an administration period, a drug holiday period, and an administration period” without providing the last drug holiday period is also included in an embodiment of the present invention. Further, in an embodiment of the present invention, a drug holiday period is a period in which drug administration is not performed for 7 or more consecutive days.

In an embodiment of the present invention, an administration period is a 14-day period or is a period including 10 days out of 14 days. 10 days out of 14 days mean any 10 days out of 14 consecutive days. The 10 days in which drug administration is performed may be 10 consecutive days or 10 non-consecutive days separated by 1-4 days in which drug administration is not performed. As an administration period, a preferred period can be selected while observing a condition of the patient.

A drug holiday period in an embodiment of the present invention is preferably a 14-day period.

The number of repetitions in the case where a 14-day administration period and a 14-day drug holiday period are repeated is not particularly limited as long as the number of repetitions is 2 or more. However, the number of repetitions is preferably 2-12, and more preferably 2-6.

In a case where an administration period of 10 days out of 14 days and a 14-day drug holiday period are repeated after an initial 14-day administration period followed by an initial 14-day drug holiday period, the number of repetitions of the administration period of 10 days out of 14 days and the 14-day drug holiday period is not particularly limited as long as the number of repetitions is 1 or more. However, the number of repetitions is preferably 1-11, and more preferably 1-5.

In another embodiment, drug administration can be repeated daily or nearly daily without providing a drug holiday period.

In administering the active ingredient, the administration route is not particularly limited, and the active ingredient may be administered orally or parenterally. Further, bolus administration and sustained administration may be possible. In the case of sustained administration, intravenous administration by infusion, transdermal administration, oral administration using a sublingual tablet, oral and intrarectal administration using a sustained-release drug product, and the like may be used. However, intravenous administration by infusion is preferable. In the case of performing bolus administration by injection or intravenous administration by infusion, for example, injectable drugs described in Japanese Patent Laid-Open Publication No. SHO 63-132833 and Japanese Patent Laid-Open Publication No. 2011-62529 may be used. The entire contents of these publications are incorporated herein by reference.

A daily dose of the active ingredient may be appropriately selected according to conditions such as age and condition of the patient. In the case of intravenous administration by infusion with providing an administration period and a drug holiday period, for an adult, an amount of 3-methyl-1-phenyl-2-pyrazolin-5-one (when the active ingredient is 3-methyl-1-phenyl-2-pyrazolin-5-one, an amount of 3-methyl-1-phenyl-2-pyrazolin-5-one; when the active ingredient is a physiologically acceptable salt of 3-methyl-1-phenyl-2-pyrazolin-5-one, an equivalent amount of 3-methyl-1-phenyl-2-pyrazolin-5-one) is preferably about 15-240 mg, more preferably about 30-180 mg, even more preferably about 60-120 mg, and particularly preferably about 60 mg. In the case where 3-methyl-1-phenyl-2-pyrazolin-5-one is administered orally, the dose is preferably pharmacokinetically substantially equivalent to the intravenous administration. A specific example is a dose for which it is recognized that a change over time of a concentration of unchanged 3-methyl-1-phenyl-2-pyrazolin-5-one of the administered 3-methyl-1-phenyl-2-pyrazolin-5-one or a physiologically acceptable salt thereof in a plasma is substantially equivalent. Examples of oral administration dosage forms include oral administration using a suspension formulation, a buccal film, a sublingual tablet, and a sustained-release drug product, and the like. For an adult, a daily amount of 3-methyl-1-phenyl-2-pyrazolin-5-one is preferably about 240-3,600 mg such as about 240 mg, about 800 mg, about 1,600 mg, about 2,400 mg, about 3,600 mg, and more preferably about 800-2,400 mg.

In the case where intravenous administration by infusion is repeated daily or nearly daily without providing a drug holiday period, for an adult, a daily amount of 3-methyl-1-phenyl-2-pyrazolin-5-one is preferably about 60 mg, about 120 mg, or about 180 mg, and particularly preferably about 60 mg, or about 120 mg.

In the case where 3-methyl-1-phenyl-2-pyrazolin-5-one is administered orally, for an adult, a daily amount of 3-methyl-1-phenyl-2-pyrazolin-5-one is preferably about 240-3,600 mg such as about 240 mg, about 800 mg, about 1,600 mg, about 2,400 mg, about 3,600 mg, and more preferably about 800-2,400 mg.

The number of doses per day during a drug administration period is not limited and a preferred number of doses per day can be selected while observing a condition of the patient. However, considering the burden of the patient, the number of doses per day is preferably 3, 2 and 1, and more preferably 1.

In the case of intravenous administration by infusion, an administration rate is desirably about 0.5-5 mg/minute, about 0.5-1 mg/minute, or about 1-5 mg/minute in the amount of 3-methyl-1-phenyl-2-pyrazolin-5-one, and, in terms of time, about 15-480 minutes, and preferably about 30-120 minutes, more preferably about 30-60 minutes, and even more preferably about 60 minutes

Regarding a drug, a treatment method or a disease progress suppression method according to an embodiment of the present invention, a patient receiving medication has at least two Features among the following identified Feature 1 to Feature 55:

1. Abnormality of gait
2. Aldolase test
3. Antinuclear antibodies (ANA) test
4. Cervical spondylosis without myelopathy
5. Creatine kinase (CK):(CPK) test
6. Cyanocobalamin (Vitamin B-12) test
7. Degeneration of cervical intervertebral disc
8. Displacement of cervical intervertebral disc without myelopathy

### 9. Dysphagia

10. Folic acid; serum test
11. Serum immunofixation electrophoresis test
12. Magnetic resonance imaging test
13. Manual therapy techniques
14. Muscle weakness
15. Needle electromyography
16. Acquired deformities of ankle and foot
17. Malaise and fatigue
18. Physical therapy evaluation
19. Serum protein electrophoretic fractionation and quantitation test
20. Erythrocyte sedimentation rate test
21. Spinal stenosis in cervical region
22. Swallowing function test
23. Therapeutic procedure for neuromuscular reeducation
24. Therapeutic procedure for therapeutic exercises
25. Thyroid stimulating hormone (TSH) test
26. Unspecified hereditary and idiopathic peripheral neuropathy
27. Nervous system disorders
28. Hereditary and degenerative nervous system conditions
29. Connective tissue disease
30. Non-traumatic joint disorders
31. Multiple sclerosis

### 32. Paraplegia

### 33. Paralysis

34. Other diagnostic nervous system procedures
35. Durable Medical Equipment (DME) and supplies
36. Physical therapy

### 37. Laryngoscopy

38. Spinal puncture
39. Treatment of speech

### 40. Riluzole

### 41. Baclofen

### 42. Pyridostigmine

### 43. Anticonvulsants

### 44. Diazepam

### 45. Hydrocodone

### 46. Propoxyphene

### 47. Sympathomimetic Agents

### 48. Glycopyrrolate

### 49. Prednisone

### 50. Pregabalin

### 51. Clonazepam

### 52. Tizanidine

### 53. Levodopa or Carbidopa

### 54. Quinine

### 55. Tolterodine

“Abnormality of gait” means that a patient has been diagnosed with a disease of “abnormality of gait” indicated by ICD-9 code 781.2 or has a symptom corresponding to the disease of “abnormality of gait.”

“Aldolase test” means that a patient has received a procedure of “aldorase” indicated by CPT code 82085 or an equivalent procedure.

“Antimuclear antibodies (ANA) test” means that a patient has received a procedure of “antimuclear antibodies (ANA)” indicated by CPT code 86038 or an equivalent procedure.

“Cervical spondylosis” means that a patient has been diagnosed with a disease of “cervical spondylosis without myelopathy” indicated by ICD-9 code 721.0 or has a symptom corresponding to the disease of “cervical spondylosis without myelopathy.”

“Cyanocobalamin (Vitamin B-12) test” means that a patient has received a procedure of “cyanocobalamin (Vitamin B-12)” indicated by CPT code 82607 or an equivalent procedure.

“Degeneration of cervical intervertebral disc” means that a patient has been diagnosed with a disease of “degeneration of cervical intervertebral disc” indicated by ICD-9 code 722.4 or has a symptom corresponding to the disease of “degeneration of cervical intervertebral disc.”

“Displacement of cervical intervertebral disc without myelopathy” means that a patient has been diagnosed with a disease of “displacement of cervical intervertebral disc without myelopathy” indicated by ICD-9 code 722.0 or has a symptom corresponding to the disease of “displacement of cervical intervertebral disc without myelopathy.”

“Dysphagia” means that a patient has been diagnosed with a disease of “dysphagia; unspecified” indicated by ICD-9 code 787.20 or has a symptom corresponding to the disease of “dysphagia; unspecified.”

“Folic acid; serum test” means that a patient has received a procedure of “folic acid; serum” indicated by CPT code 82746 or an equivalent procedure.

“Serum immunofixation electrophoresis test” means that a patient has received a procedure of “immunofixation electrophoresis; serum” indicated by CPT code 86334 or an equivalent procedure.

“Magnetic resonance imaging test” means that a patient has received a procedure of “injection; gadolinium-based magnetic resonance contrast agent; not otherwise specified (nos); per ml” indicated by CPT code A9579, a procedure of “magnetic resonance (eg; proton) imaging; brain (including brain stem); without contrast material” indicated by CPT code 70551, a procedure of “magnetic resonance (eg; proton) imaging; brain (including brain stem); without contrast material; followed by contrast material(s) and further sequences” indicated by CPT code 70553, a procedure of “magnetic resonance (eg; proton) imaging; spinal canal and contents; cervical; without contrast material” indicated by CPT code 72141, a procedure of “magnetic resonance (eg; proton) imaging; spinal canal and contents; lumbar; without contrast material” indicated by CPT code 72148, or a procedure of “magnetic resonance (eg; proton) imaging; spinal canal and contents; without contrast material; followed by contrast material(s) and further sequences; cervical” indicated by CPT code 72156, or a procedure equivalent to these procedures.

“Manual therapy techniques” means that a patient has received a procedure of “manual therapy techniques (eg; mobilization/manipulation; manual lymphatic drainage; manual traction); 1 or more regions; each 15 minutes” indicated by CPT code 97140 or an equivalent procedure.

“Muscle weakness” means that a patient has been diagnosed with a disease of “muscle weakness (generalized)” indicated by ICD-9 code 728.87 or has a symptom corresponding to the disease of “muscle weakness (generalized).”

“Needle electromyography” means that a patient has received a procedure of “needle electromyography; 1 extremity with or without related paraspinal areas” indicated by CPT code 95860 or a procedure of “needle electromyography; 2 extremities with or without related paraspinal areas” indicated by CPT code 95861, or a procedure equivalent to these procedures.

“Acquired deformities of ankle and foot” means that a patient has been diagnosed with a disease of “other acquired deformities of ankle and foot” indicated by ICD-9 code 736.79 or has a symptom corresponding to the disease of “other acquired deformities of ankle and foot.”

“Malaise and fatigue” means that a patient has been diagnosed with a disease of “other malaise and fatigue” indicated by ICD-9 code 780.79 or has a symptom corresponding to the disease of “other malaise and fatigue.”

“Physical therapy evaluation” means that a patient has received a procedure of “physical therapy evaluation” indicated by CPT code 97001 or an equivalent procedure.

“Serum protein electrophoretic fractionation and quantitation test” means that a patient has received a procedure of “protein; electrophoretic fractionation and quantitation; serum” indicated by CPT code 84165 or an equivalent procedure.

“Erythrocyte sedimentation rate test” means that a patient has received a procedure of “sedimentation rate; erythrocyte; automated” indicated by CPT code 85652 or a procedure of “sedimentation rate; erythrocyte; non-automated” indicated by CPT code 85651, or a procedure equivalent to these procedures.

“Spinal stenosis in cervical region” means that a patient has been diagnosed with a disease of “spinal stenosis in cervical region” indicated by ICD-9 code 723.0 or has a symptom corresponding to the disease of “spinal stenosis in cervical region.”

“Swallowing function; with cineradiography/videoradiography” means that a patient has received a procedure of “swallowing function; with cineradiography/videoradiography” indicated by CPT code 74230 or an equivalent procedure.

“Therapeutic procedure for neuromuscular reeducation of movement; balance; coordination; kinesthetic sense; posture; and/or proprioception for sitting and/or standing activities” means that a patient has received a procedure of “therapeutic procedure; 1 or more areas; each 15 minutes; neuromuscular reeducation of movement; balance; coordination; kinesthetic sense; posture; and/or proprioception for sitting and/or standing activities” indicated by CPT code 97112 or an equivalent procedure.

“Therapeutic procedure for therapeutic exercises to develop strength and endurance; range of motion and flexibility” means that a patient has received a procedure of “therapeutic procedure; 1 or more areas; each 15 minutes; therapeutic exercises to develop strength and endurance; range of motion and flexibility” indicated by CPT code 97110, or an equivalent procedure.

“Thyroid stimulating hormone (TSH) test” means that a patient has received a procedure of “thyroid stimulating hormone (TSH)” indicated by CPT code 84443 or an equivalent procedure.

“Unspecified hereditary and idiopathic peripheral neuropathy” means that a patient has been diagnosed with a disease of “unspecified hereditary and idiopathic peripheral neuropathy” indicated by ICD-9 code 356.9 or has a symptom corresponding to the disease of “unspecified hereditary and idiopathic peripheral neuropathy.”

“Nervous system disorders” means that a patient has been diagnosed with a disease of “other nervous system disorders” or has a symptom corresponding to the disease of “other nervous system disorders.”

“Hereditary and degenerative nervous system conditions” means that a patient has been diagnosed with a disease of “other hereditary and degenerative nervous system conditions” or has a symptom corresponding to the disease of “other hereditary and degenerative nervous system conditions.”

“Connective tissue disease” means that a patient has been diagnosed with a disease of “connective tissue disease” or has a symptom corresponding to the disease of “connective tissue disease.”

“Non-traumatic joint disorders” means that a patient has been diagnosed with a disease of “other non-traumatic joint disorders” or has a symptom corresponding to the disease of “other non-traumatic joint disorders.”

“Multiple sclerosis” means that a patient has been diagnosed with a disease of “multiple sclerosis” or has a symptom corresponding to the disease of “multiple sclerosis.”

“Paraplegia” means that a patient has been diagnosed with a disease of “paraplegia” or has a symptom corresponding to the disease of “paraplegia.”

“Paralysis” means that a patient has been diagnosed with a disease of “paralysis” or has a symptom corresponding to the disease of “paralysis.”

“Other diagnostic nervous system procedures” means that a patient has received a procedure of “other diagnostic nervous system procedures” or an equivalent procedure.

“Durable Medical Equipment (DME) and supplies” means that a patient has received a procedure of “Durable Medical Equipment (DME) and supplies” or an equivalent procedure.

“Physical therapy” means that a patient has received a procedure of “physical therapy” or an equivalent procedure.

“Laryngoscopy” means that a patient has received a procedure of “laryngoscopy” or an equivalent procedure.

“Spinal puncture” means that a patient has received a procedure of “spinal puncture” or an equivalent procedure.

“Treatment of speech” means that a patient has received a procedure of “treatment of speech” or an equivalent procedure.

“Riluzole” means that a patient has been prescribed a medication containing “Riluzole” as an active ingredient.

“Baclofen” means that a patient has been prescribed “Baclofen.”

“Pyridostigmine” means that a patient has been prescribed a medication containing “Pyridostigmine” as an active ingredient.

“Anticonvulsants” means that a patient has been prescribed one or more medications containing active ingredients classified as “Anticonvulsants.”

“Diazepam” means that a patient has been prescribed a medication containing “Diazepam” as an active ingredient.

“Hydrocodone” means that a patient has been prescribed a medication containing “Hydrocodone” as an active ingredient.

“Propoxyphene” means that a patient has been prescribed a medication containing “Propoxyphene” as an active ingredient.

“Propoxyphene” means that a patient has been prescribed a medication containing “Sympathomimetic Agents” as active ingredients.

“Glycopyrrolate” means that a patient has been prescribed a medication containing “Glycopyrrolate” as an active ingredient.

“Prednisone” means that a patient has been prescribed a medication containing “Prednisone” as an active ingredient.

“Pregabalin” means that a patient has been prescribed a medication containing “Pregabalin” as an active ingredient.

“Clonazepam” means that a patient has been prescribed a medication containing “Clonazepam” as an active ingredient.

“Tizanidine” means that a patient has been prescribed a medication containing “Tizanidine” as an active ingredient.

“Levodopa or Carbidopa” means that a patient has been prescribed a medication containing “Levodopa” as an active ingredient or a medication containing “Carbidopa” as an active ingredient.

“Quinine” means that a patient has been prescribed a medication containing “Quinine” as an active ingredient.

“Tolterodine” means that a patient has been prescribed a medication containing “Tolterodine” as an active ingredient.

A patient having a feature identified by the present invention is highly likely to be an ALS patient as compared to other patients, and it is expected that it is particularly effective to administer a medication containing 3-methyl-1-phenyl-2-pyrazolin-5-one or a physiologically acceptable salt thereof to the patient.

Further, regarding a drug, a treatment method or a disease progress suppression method according to an embodiment of the present invention, a patient receiving medication may meet one or more of the following Features:
Skin disorders
Any changes in speech
Office visit to: physical therapy, neurologist, orthopedic surgeon, gastroenterologist, or otolaryngologist
Unusual increase in healthcare resource utilization (i.e. increase doctor visit, procedures (MM, EMG) new diagnosis, prescriptions)
Unusually higher healthcare utilization

A change in speech notable, noted, recognizable and/or recognized by a medical profession such as a medical doctor, a nurse, a therapist, and a health care provider.

An unusual increase in healthcare resource utilization notable, noted, recognizable or recognized by a medical profession such as a medical doctor, a nurse, a therapist, and a health care provider.

An unusually higher healthcare utilization notable, noted, recognizable or recognized by a medical profession such as a medical doctor, a nurse, a therapist, and a health care provider.

Regarding a drug, a treatment method or a disease progress suppression method according to an embodiment of the present invention, during a certain time period before receiving initial administration of 3-methyl-1-phenyl-2-pyrazolin-5-one or a physiologically acceptable salt thereof, a patient receiving medication preferably has at least two Features among the Feature 1 to Feature 55. The time period may be a certain time period within 120 months before receiving the initial administration. More preferably, the time period is a certain time period within 96 months, 72 months, 60 months, 48 months, 36 months, 24 months, or 12 months before receiving the initial administration.

The start and end of the time period are not particularly limited as long as the time period is within 120 months before receiving the initial administration. The time period may include one time period or two or more time periods, and lengths of the time periods may be the same or different. The number of the time periods is not particularly limited, but is preferably 1-20, more preferably 1-15, and even more preferably any one of 1, 2, 3, 4, 5, 6, 7, 8, 9, and 10. Lengths of the time periods are not particularly limited, but can be 1 month, 2 months, 3 months, 6 months, 9 months, 12 months, 15 months, 18 months, 24 months, 30 months, 36 months, 48 months, 60 months, 72 months, 96 months, and 120 months. In some embodiments, before receiving initial administration, in at least one of periods of 0 to 3 months, 3 to 6 months, 6 to 9 months, 9 to 12 months, 12 to 18 months, 18 to 24 months, 24 to 36 months, and 36 to 48 months, a patient receiving medication has at least two Features among the Feature 1 to Feature 55.

In another embodiment of the present invention, regarding a drug, a treatment method or a disease progress suppression method, a patient receiving medication has at least one pair of Features identified in the following pair 1 to pair 46:

### 1. Feature (1) and Feature (14)

### 2. Feature (1) and Feature (18)

### 3. Feature (1) and Feature (30)

### 4. Feature (2) and Feature (5)

### 5. Feature (3) and Feature (5)

### 6. Feature (3) and Feature (6)

### 7. Feature (3) and Feature (18)

### 8. Feature (3) and Feature (31)

### 9. Feature (4) and Feature (14)

### 10. Feature (5) and Feature (6)

### 11. Feature (5) and Feature (18)

### 12. Feature (5) and Feature (20)

### 13. Feature (5) and Feature (25)

### 14. Feature (5) and Feature (26)

### 15. Feature (5) and Feature (31)

### 16. Feature (6) and Feature (10)

### 17. Feature (6) and Feature (13)

### 18. Feature (6) and Feature (14)

### 19. Feature (6) and Feature (16)

### 20. Feature (6) and Feature (18)

### 21. Feature (6) and Feature (20)

### 22. Feature (6) and Feature (24)

### 23. Feature (6) and Feature (26)

### 24. Feature (6) and Feature (31)

### 25. Feature (7) and Feature (14)

### 26. Feature (8) and Feature (14)

### 27. Feature (9) and Feature (28)

### 28. Feature (11) and Feature (13)

### 29. Feature (12) and Feature (14)

### 30. Feature (13) and Feature (16)

### 31. Feature (14) and Feature (15)

### 32. Feature (14) and Feature (18)

### 33. Feature (14) and Feature (20)

### 34. Feature (14) and Feature (22)

### 35. Feature (14) and Feature (27)

### 36. Feature (15) and Feature (21)

### 37. Feature (17) and Feature (30)

### 38. Feature (18) and Feature (19)

### 39. Feature (18) and Feature (21)

### 40. Feature (18) and Feature (22)

### 41. Feature (18) and Feature (30)

### 42. Feature (18) and Feature (31)

### 43. Feature (18) and Feature (32)

### 44. Feature (21) and Feature (30)

### 45. Feature (23) and Feature (30)

### 46. Feature (29) and Feature (30)

Feature (1) is “Abnormality of gait”;

### Feature (2) is “Aldolase”;

Feature (3) is “Antinuclear antibodies (ANA)”;
Feature (4) is “Cervical spondylosis without myelopathy”;
Feature (5) is “Creatine kinase (CK); (CPK); total”;

### Feature (6) is “Cyanocobalamin (Vitamin B-12)”;

Feature (7) is “Degeneration of cervical intervertebral disc”;
Feature (8) is “Displacement of cervical intervertebral disc without myelopathy”;
Feature (9) is “Dysphagia; unspecified”;
Feature (10) is “Folic acid; serum”;
Feature (11) is “Injection; gadolinium-based magnetic resonance contrast agent; not otherwise specified (nos); per ml”;
Feature (12) is “Magnetic resonance (eg; proton) imaging; brain (including brain stem); without contrast material”;
Feature (13) is “Magnetic resonance (eg; proton) imaging; brain (including brain stem); without contrast material; followed by contrast material (s) and further sequences”;
Feature (14) is “Magnetic resonance (eg; proton) imaging; spinal canal and contents; cervical; without contrast material”;
Feature (15) is “Magnetic resonance (eg; proton) imaging; spinal canal and contents; lumbar; without contrast material”;
Feature (16) is “Magnetic resonance (eg; proton) imaging; spinal canal and contents; without contrast material; followed by contrast material (s) and further sequences; cervical”;
Feature (17) is “Manual therapy techniques (eg; mobilization/manipulation; manual lymphatic drainage; manual traction); 1 or more regions; each 15 minutes”;
Feature (18) is “Muscle weakness (generalized)”;
Feature (19) is “Needle electromyography; 1 extremity with or without related paraspinal areas”;
Feature (20) is “Needle electromyography; 2 extremities with or without related paraspinal areas”;
Feature (21) is “Other acquired deformities of ankle and foot”;
Feature (22) is “Other malaise and fatigue”;
Feature (23) is “Physical therapy evaluation”;
Feature (24) is “Protein; electrophoretic fractionation and quantitation; serum”;
Feature (25) is “Sedimentation rate; erythrocyte; automated”;
Feature (26) is “Sedimentation rate; erythrocyte; non-automated”;
Feature (27) is “Spinal stenosis in cervical region”;
Feature (28) is “Swallowing function; with cineradiography/videoradiography”;
Feature (29) is “Therapeutic procedure; 1 or more areas; each 15 minutes; neuromuscular reeducation of movement; balance; coordination; kinesthetic sense; posture; and/or proprioception for sitting and/or standing activities”;
Feature (30) is “Therapeutic procedure; 1 or more areas; each 15 minutes; therapeutic exercises to develop strength and endurance; range of motion and flexibility”;
Feature (31) is “Thyroid stimulating hormone (TSH)”;
Feature (32) is “Unspecified hereditary and idiopathic peripheral neuropathy”.

Further, regarding a drug, a treatment method or a disease progress suppression method according to yet another embodiment, a patient receiving medication has at least 3 Features, preferably at least 4 Features, and more preferably at least 5 Features among the following identified Features:

1. Malaise and fatigue, or Muscle weakness

2. Non-traumatic joint disorder or Acquired deformities of ankle and foot

3. Connective tissue disease

4. Skin disorder

5. Nervous system disorder

6. Any change in speech

7. Office visit to: Physical therapy, Neurologist, Orthopedic surgeon, Gastroenterologist, or Otolaryngologist

8. Magnetic resonance imaging test, or Needle electromyography

9. Riluzole, Baclofen, Pyridostigmine, Anticonvulsants

10. Unusual increase in healthcare resource utilization

11. Creatine kinase (CK):(CPK) test, Cyanocobalamin (Vitamin B-12) test, or Antinuclear antibodies (ANA) test

Regarding a drug, a treatment method or a disease progress suppression method of another embodiment, before receiving initial administration of 3-methyl-1-phenyl-2-pyrazolin-5-one or a physiologically acceptable salt thereof, in at least one of periods of 0 to 3 months, 3 to 6 months, 6 to 9 months, 9 to 12 months, 12 to 18 months, 18 to 24 months, 24 to 36 months, and 36 to 48 months, a patient receiving medication has at least 3 Features, preferably at least 4 Features, and more preferably at least 5 Features among the following identified Features:

1. Malaise and fatigue, or Muscle weakness

2. Non-traumatic joint disorder or Acquired deformities of ankle and foot

3. Connective tissue disease

4. Skin disorder

5. Nervous system disorder

6. Any change in speech

7. Office visit to: Physical therapy, Neurologist, Orthopedic surgeon, Gastroenterologist, or Otolaryngologist

8. Magnetic resonance imaging test, or Needle electromyography

9. Riluzole, Baclofen, Pyridostigmine, Anticonvulsants

10. Unusual increase in healthcare resource utilization

11. Creatine kinase (CK):(CPK) test, Cyanocobalamin (Vitamin B-12) test, or Antinuclear antibodies (ANA) test

In this embodiment, it is also possible that a numerical value between 0 and 1 is appropriately selected for each of the identified Features and weighting is performed for each of the Features. In this case, for a patient receiving medication, before receiving initial administration of 3-methyl-1-phenyl-2-pyrazolin-5-one or a physiologically acceptable salt thereof, in at least one of periods of 0 to 3 months, 3 to 6 months, 6 to 9 months, 9 to 12 months, 12 to 18 months, 18 to 24 months, 24 to 36 months, and 36 to 48 months, a sum of the numerical values of the above-identified Features is 3 or more, preferably 4 or more, and more preferably 5 or more.

Further, regarding a drug, a treatment method or a disease progress suppression method according to another embodiment, before receiving initial administration of 3-methyl-1-phenyl-2-pyrazolin-5-one or a physiologically acceptable salt thereof, in periods of 0 to 3 months, 3 to 6 months, 6 to 9 months, 9 to 12 months, 12 to 18 months, 18 to 24 months, 24 to 36 months, and 36 to 48 months, a sum of numbers of Features that a patient receiving medication has among the following identified Features is at least 15, preferably at least 20, and more preferably at least 25.

1. Malaise and fatigue, or Muscle weakness

2. Non-traumatic joint disorder or Acquired deformities of ankle and foot

3. Connective tissue disease

4. Skin disorder

5. Nervous system disorder

6. Any change in speech

7. Office visit to: Physical therapy, Neurologist, Orthopedic surgeon, Gastroenterologist, or Otolaryngologist

8. Magnetic resonance imaging test, or Needle electromyography

9. Riluzole, Baclofen, Pyridostigmine, Anticonvulsants

10. Unusual increase in healthcare resource utilization

11. Creatine kinase (CK):(CPK) test, Cyanocobalamin (Vitamin B-12) test, or Antinuclear antibodies (ANA) test

Regarding the drug, the treatment method or the disease progress suppression method of this embodiment, it is also possible that a numerical value between 0 and 1 is appropriately selected for each of the identified Features and weighting is performed for each of the Features. In this case, for a patient receiving medication, before receiving initial administration of 3-methyl-1-phenyl-2-pyrazolin-5-one or a physiologically acceptable salt thereof, in periods of 0 to 3 months, 3 to 6 months, 6 to 9 months, 9 to 12 months, 12 to 18 months, 18 to 24 months, 24 to 36 months, and 36 to 48 months, a sum of the numerical values of the above-identified Features is 15 or more, preferably 20 or more, and more preferably 25 or more.

Regarding a drug, a treatment method or a disease progress suppression method according to another embodiment, a step may be provided in which a patient having an identified Feature is selected before receiving initial administration of 3-methyl-1-phenyl-2-pyrazolin-5-one or a physiologically acceptable salt thereof.

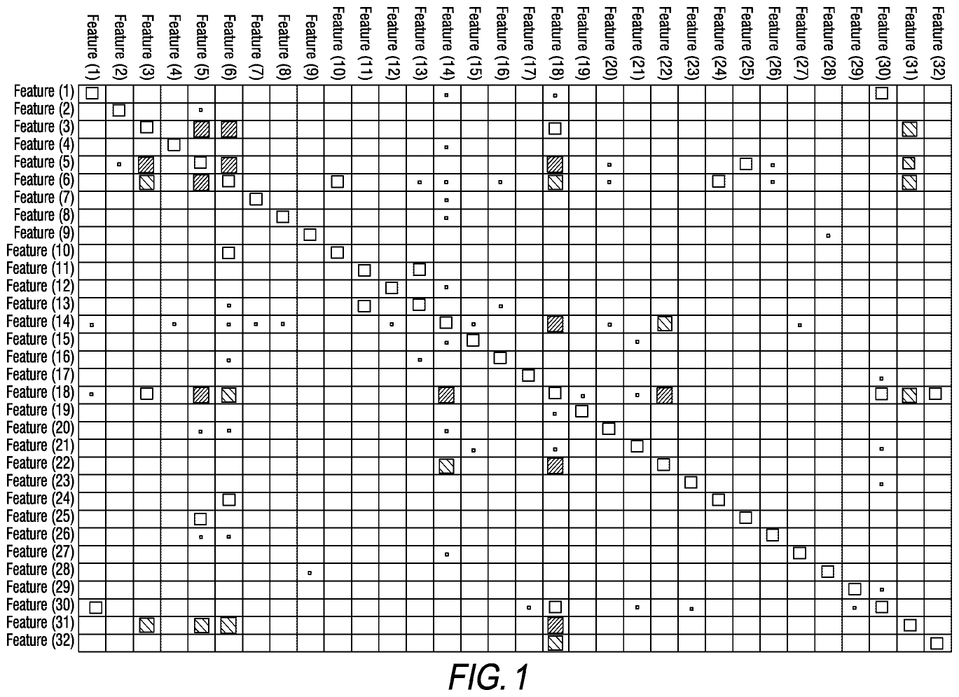

FIG. 1 illustrates top 20 two-Feature combinations based on mutual information rank and values in periods of three to six months prior to patients are diagnosed as having ALS. Top 20 two-Feature combinations in three to six month periods prior to diagnosis plotted in Feature-to-Feature heat maps. Each axis lists all single Features included in combinations. Block representations of mutual information values of the Feature combinations are plotted at the Feature intersections on grid, with larger and darker blocks representing higher mutual information values.

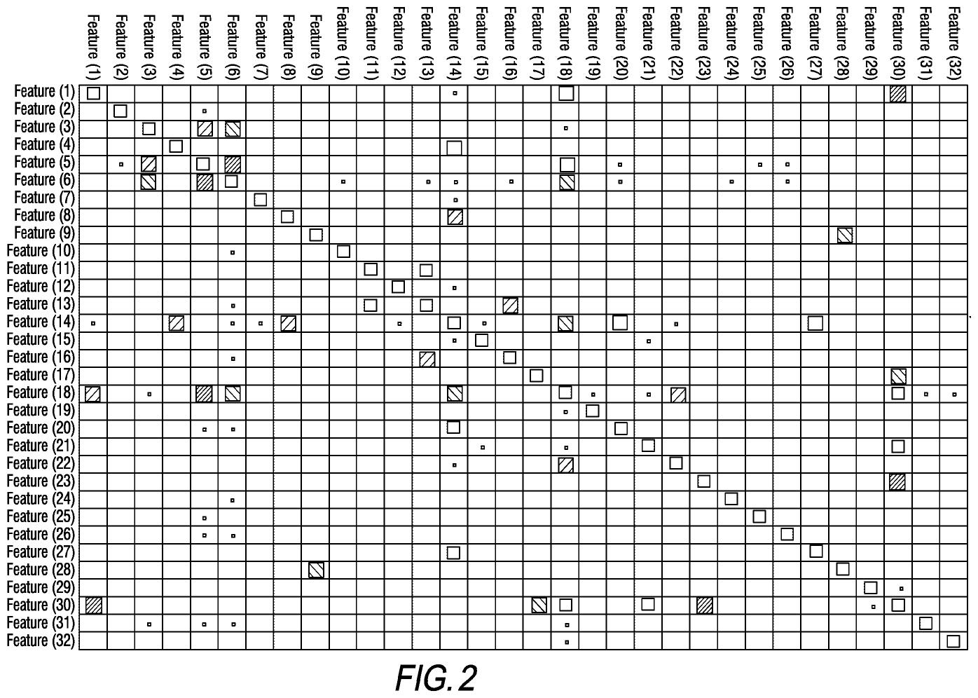

FIG. 2 illustrates top 20 two-Feature combinations based on mutual information rank and values in periods of six to nine months prior to patients are diagnosed as having ALS. Top 20 two-Feature combinations in six to nine month periods prior to diagnosis plotted in Feature-to-Feature heat maps. Each axis lists all single Features included in combinations. Block representations of mutual information values of the Feature combinations are plotted at the Feature intersections on grid, with larger and darker blocks representing higher mutual information values.

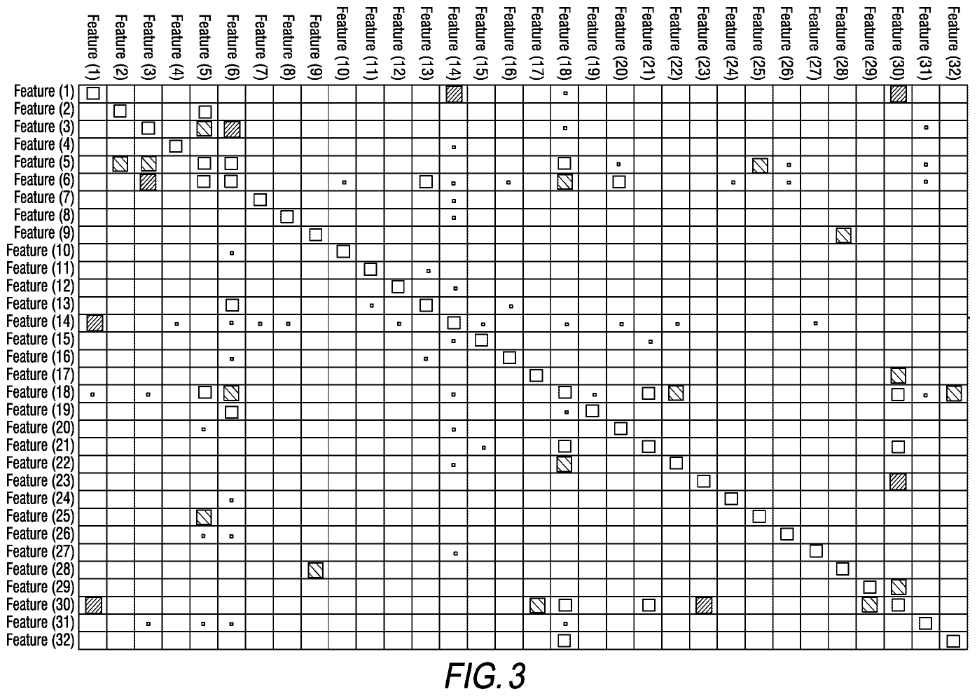

FIG. 3 illustrates top 20 two-Feature combinations based on mutual information rank and values in periods of nine to twelve months prior to patients are diagnosed as having ALS. Top 20 two-Feature combinations in nine to twelve month periods prior to diagnosis plotted in Feature-to-Feature heat maps. Each axis lists all single Features included in combinations. Block representations of mutual information values of the Feature combinations are plotted at the Feature intersections on grid, with larger and darker blocks representing higher mutual information values.

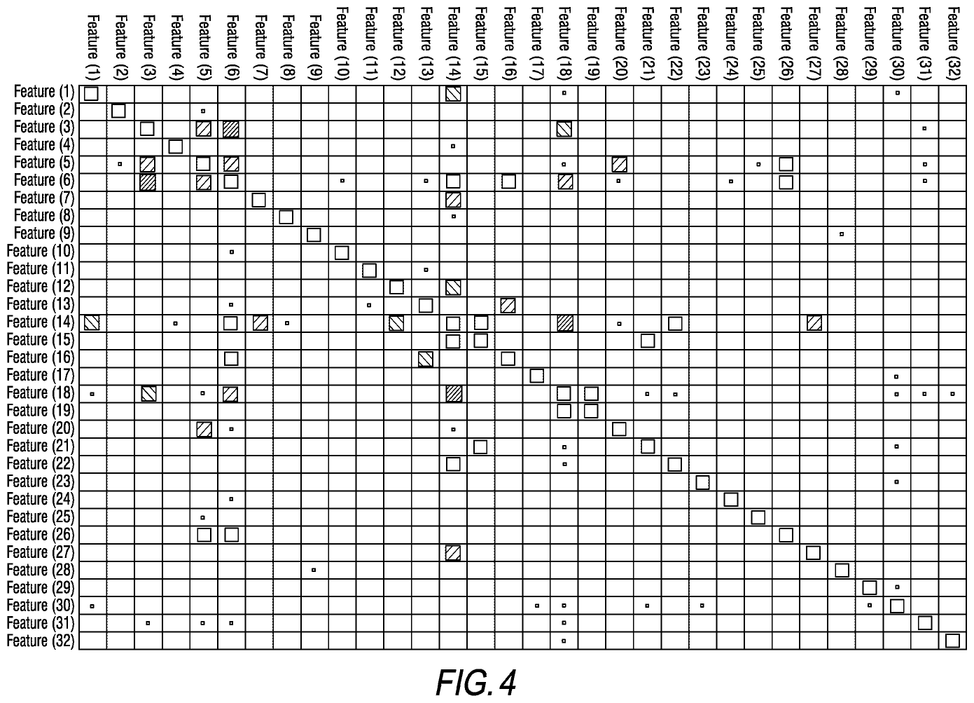

FIG. 4 illustrates top 20 two-Feature combinations based on mutual information rank and values in periods of twelve to eighteen months prior to patients are diagnosed as having ALS. Top 20 two-Feature combinations in twelve to eighteen month periods prior to diagnosis plotted in Feature-to-Feature heat maps. Each axis lists all single Features included in combinations. Block representations of mutual information values of the Feature combinations are plotted at the Feature intersections on grid, with larger and darker blocks representing higher mutual information values.

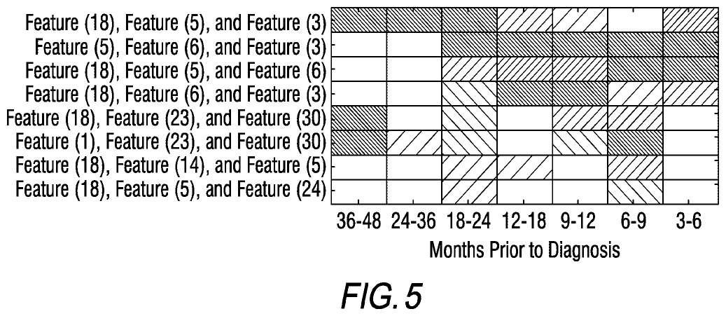

FIG. 5 illustrates selected 3 Feature combinations by mutual information rank in periods of thirty-six to forty-eight months, twenty-four to thirty-six months, eighteen to twenty-four months, twelve to eighteen months, nine to twelve months, six to nine months, and three to six months prior to patients are diagnosed as having ALS.

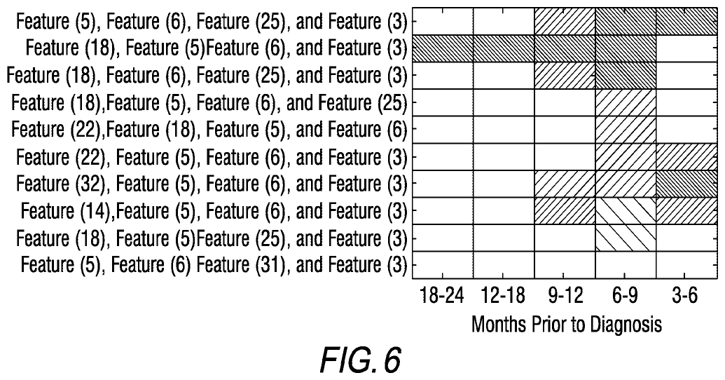

FIG. 6 illustrates selected 4 Feature combinations by mutual information rank in periods of eighteen to twenty-four months, twelve to eighteen months, nine to twelve months, six to nine months, and three to six months prior to patients are diagnosed as having ALS.

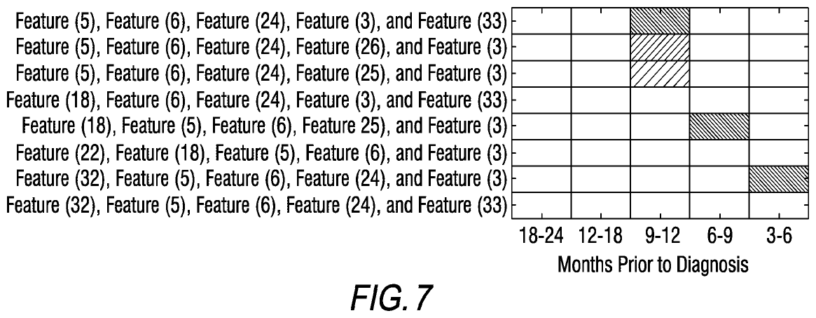

FIG. 7 illustrates selected 5 Feature combinations by mutual information rank in periods of eighteen to twenty-four months, twelve to eighteen months, nine to twelve months, six to nine months, and three to six months prior to patients are diagnosed as having ALS.

In FIG. 7, Feature (1) to Feature (32) are the same as above.

Feature (33) is “Immunofixation electrophoresis; serum”

International Publication No. WO 2002/034264 describes that 3-methyl-1-phenyl-2-pyrazolin-5-one is useful for treating ALS. However, the dosage form, the dose, the number of doses and the like of this compound to an ALS patient are not specifically disclosed. International Publication No. WO 2005/075434 describes a drug for treating and/or suppressing progress of amyotrophic lateral sclerosis or a symptom caused by amyotrophic lateral sclerosis, which includes 3-methyl-1-phenyl-2-pyrazolin-5-one as an active ingredient, where a drug holiday period of one or more days is established one or more times in a period of treating and/or suppressing progress of the disease. Further, a method has been reported in which 30 mg of 3-methyl-1-phenyl-2-pyrazolin-5-one is administered to an ALS patient by infusion for 14 days followed by administering it for 10 days per month (Neurotherapy, 2003, Vol. 20, No. 5, pages 557-564). A method for treating ALS has been reported in which 3-methyl-1-phenyl-2-pyrazolin-5-one is administered to patients with a particularly high therapeutic effect among ALS patients in need of treatment.

Diagnostic criteria for ALS include EL Escorial diagnostic criteria, EL Escorial revised Airlie House diagnostic criteria, Awaji diagnostic criteria, and the like.

It has been reported that an average time period from appearance of an initial symptom of ALS to receiving a diagnosis of ALS is one year. There are multiple factors for the delay in diagnosis of ALS. The first is that a period from the appearance of the initial symptom to a first visit to a doctor is long. The second is that an early symptom of ALS is similar to that of other diseases. On average, three doctors are visited by an ALS patient from the first doctor visit to when a final diagnosis is received (Paganoni S, et al. Amyotroph Lateral Scler Frontotemporal Degener. 2014; 15 (5-6), 453). Therefore, new treatment method and suppression method are necessary for shortening a period from appearance of an initial symptom of ALS to when a final diagnosis is received and for treating an ALS patient at an early stage or suppressing progress of ALS of an ALS patient at an early stage.

ALSFRS-R is a severity index for an ALS patient and includes a total of 12 evaluation items regarding motor dysfunction of limbs, bulbar dysfunction, and respiratory dysfunction. For example, in clinical trials, by comparing an ALSFRS-R score before start of administration of an active ingredient to a patient, an ALSFRS-R score of a certain period after the start of the administration, and/or an ALSFRS-R score after the administration, an effect of the active ingredient may be confirmed.

An example of a method for synthesizing 3-methyl-1-phenyl-2-pyrazolin-5-one, which is the active ingredient of the present invention, is a manufacturing method described in European Patent Publication No. 208874 (or Japanese Patent Publication No. HEI 5-31523). The entire contents of these publications are incorporated herein by reference.

Examples of the active ingredient of the drug in the present invention include 3-methyl-1-phenyl-2-pyrazolin-5-one, a physiologically acceptable salt thereof, a hydrate thereof, and a solvate thereof. Examples of physiologically acceptable salts include salts with mineral acids such as hydrochloric acid, hydrobromide, and phosphoric acid; salts with organic acids such as methanesulfonic acid, p-toluenesulfonic acid, acetic acid, oxalic acid, citric acid, malic acid, and maric acid; salts with alkali metals such as sodium, and potassium; salts with alkaline earth metals such as magnesium; and salts with amines such as ammonia, ethanolamine, and 2-amino-2-methyl-1-propanol. In addition, the type of salt is not particularly limited as long as the salt is physiologically acceptable.

3-methyl-1-phenyl-2-pyrazolin-5-one or a salt thereof, which is the active ingredient of the drug of the present invention, may be directly administered to a patient. However, it is preferable to provide a drug product obtained by adding the active ingredient and pharmacologically and pharmaceutically acceptable additives.

As the pharmacologically and pharmaceutically acceptable additives, for example, an excipient, a disintegrating agent or a disintegration aid, a binding agent, a lubricant, a coating agent, a pigment, a diluent, a base, a solubilizer or a solubilizing agent, an isotonizing agent, a pH regulator, a stabilizer, a propellant, an adhesive, and the like may be used. Examples of drug products suitable for oral administration include tablets, capsules, powders, fine granules, granules, liquid drugs, syrups, and the like. Examples of drug products suitable for parenteral administration include injectable drugs, drops, adhesive skin patches, suppositories, and the like.

As additives for drug products suitable for oral administration, for example, the following additives may be used: excipients such as glucose, lactose, D-mannitol, starch, or crystalline cellulose; disintegrating agents or disintegration aids such as carboxymethylcellulose, starch, or carboxymethylcellulose calcium; binding agents such as hydroxypropylcellulose, hydroxypropylmethylcellulose, polyvinylpyrrolidone, or gelatin; lubricants such as magnesium stearate or talc; coating agents such as hydroxypropylmethylcellulose, white sugar, polyethylene glycol or titanium oxide; and bases such as vaseline, liquid paraffin, polyethylene glycol, gelatin, kaolin, glycerin, purified water, or hard fat.

For drug products suitable for injection or infusion, the following additives for drug products may be used: solubilizers or solubilizing agents, which are capable of forming aqueous injectable drugs or injectable drugs dissolvable when used, such as distilled water for injection, physiological saline, propylene glycol and the like; isotonizing agents such as glucose, sodium chloride, D-mannitol, glycerin and the like; pH regulators such as inorganic acids, organic acids, inorganic bases or organic bases; and the like.

A cerebral protective agent (injectable drug) containing 3-methyl-1-phenyl-2-pyrazolin-5-one as an active ingredient has already been used clinically (generic name: “Edaravone”; trade name: “Radicut (registered trademark),” “Radicava (registered trademark)”: manufactured by and commercially available from Mitsubishi Tanabe Pharma Co., Ltd.). Therefore, as the 3-methyl-1-phenyl-2-pyrazolin-5-one used in the drug and method of the present invention, the above drug products may be directly used.

### EXAMPLES

In the following, the embodiments of the present invention are further described based on Examples. However, the scope of the present invention is not limited to the following Examples.

### Method

The TruvenMarketScan® database, containing patient-level claims for 170+ million patients, was used without any code pre-selection for this analysis.

Patients with ALS were identified using ICD-9 code 335.20 and ICD-10 code G12.21. Patient demographics were reported for a nationwide set of patients with an ALS ICD-9 or ICD-10 code between January 2010 and June 2016. Patients from the full nationwide adjudicated claims database covering 2006 through 2014 with an ALS ICD-9 code and a minimum of 1 year of adjudicated claims history prior to ALS diagnosis were included in the frequency analyses. Patients from the full nationwide adjudicated claims database covering 2006 through 2014 with an ALS ICD-9 code and a minimum of 5 years of adjudicated claims history prior to ALS diagnosis were included in the disease progression analysis.

This analysis utilized 2 data ranking methods: a frequency method and a mutual information (MI) method; the MI measure was used to quantify the statistical relevance of every feature in MarketScan® to a future ALS diagnosis in the US; the relative frequency of pertinent events was computed to rank the differentiating features

In these analyses, patients from the full national data set were included (n=13,882).

Features considered included diagnosis codes, procedure codes, medications, standard provider types, and standard care facility types.

An ensembled suite of classifiers developed through machine learning techniques were applied to the MarketScan® claims database to optimize the selection and ranking of ALS diagnosis predictors.

Diagnosis predictors were derived from the differentiating features selected by mutual information and ranked using machine learning techniques.

Features were analyzed in combination in addition to individual features. Combinations of up to five of drugs, procedures and diagnosis. Combinations of same feature types (drug 1+drug 2, procedure 1+procedure 2) as well as multiple feature types (procedure+diagnosis)

Diagnosis predictors were specifically looked for within the following time brackets: 3, 6, 9, 12, 18, 24, 36, 48, and 60 months before the initial ALS diagnosis.

Regarding mutual information (MI) values of Feature 1-Feature 2 combinations in periods of 3-6 months, 6-9 months, 9-12 months and 12-18 months before the initial ALS diagnosis, top 20 MI values in each of these periods are shown in the following table.

| | | Mutual Information (MI) Value of | | | | Number of |
|---|---|---|---|---|---|---|
| | | Feature 1-Feature 2 Combination | | | | Top 20 |
| Feature 1 | Feature 2 | 3-6 Months | 6-9 Months | 9-12 Months | 12-18 Months | Appearances |
| Feature (1) | Feature (14) | | | 0.00096 | 0.00137 | 2 |
| Feature (1) | Feature (18) | | 0.00128 | | | 1 |
| Feature (1) | Feature (30) | 0.00321 | 0.00178 | 0.00105 | | 3 |
| Feature (2) | Feature (5) | | | 0.00086 | | 1 |
| Feature (3) | Feature (5) | 0.00462 | 0.00135 | 0.00088 | 0.00122 | 4 |
| Feature (3) | Feature (6) | 0.00437 | 0.00152 | 0.00109 | 0.00161 | 4 |
| Feature (3) | Feature (18) | 0.00326 | | | 0.00144 | 2 |
| Feature (3) | Feature (31) | 0.00383 | | | | 1 |
| Feature (4) | Feature (14) | | 0.00129 | | | 1 |
| Feature (5) | Feature (2) | | | 0.00086 | | 1 |
| Feature (5) | Feature (3) | 0.00462 | 0.00135 | 0.00088 | 0.00122 | 4 |
| Feature (5) | Feature (6) | 0.00424 | 0.00172 | 0.00082 | 0.00129 | 4 |
| Feature (5) | Feature (18) | 0.00423 | 0.00160 | 0.00082 | | 3 |
| Feature (5) | Feature (20) | | | | 0.00124 | 1 |
| Feature (5) | Feature (25) | 0.00318 | | 0.00087 | | 2 |
| Feature (5) | Feature (26) | | | | 0.00112 | 1 |
| Feature (5) | Feature (31) | 0.00356 | | | | 1 |
| Feature (6) | Feature (3) | 0.00437 | 0.00152 | 0.00109 | 0.00161 | 4 |
| Feature (6) | Feature (5) | 0.00424 | 0.00172 | 0.00082 | 0.00129 | |
| Feature (6) | Feature (10) | 0.00302 | | | | 1 |
| Feature (6) | Feature (13) | | | 0.00078 | | 1 |
| Feature (6) | Feature (14) | | | | 0.00115 | 1 |
| Feature (6) | Feature (16) | | | | 0.00113 | 1 |
| Feature (6) | Feature (18) | 0.00392 | 0.00148 | 0.00092 | 0.00127 | 4 |
| Feature (6) | Feature (20) | | | 0.00080 | | 1 |
| Feature (6) | Feature (24) | 0.00340 | | | | 1 |
| Feature (6) | Feature (26) | | | | 0.00112 | 1 |
| Feature (6) | Feature (31) | 0.00383 | | | | 1 |
| Feature (7) | Feature (14) | | | | 0.00120 | 1 |
| Feature (8) | Feature (14) | | 0.00133 | | | 1 |
| Feature (9) | Feature (28) | | 0.00151 | 0.00092 | | 2 |
| Feature (10) | Feature (6) | 0.00302 | | | | 1 |
| Feature (11) | Feature (13) | 0.00304 | 0.00122 | | | 2 |
| Feature (12) | Feature (14) | | | | 0.00145 | 1 |
| Feature (13) | Feature (6) | | | 0.00078 | | 1 |
| Feature (13) | Feature (11) | 0.00304 | 0.00122 | | | 2 |
| Feature (13) | Feature (16) | | 0.00133 | | 0.00126 | 2 |
| Feature (14) | Feature (1) | | | 0.00096 | 0.00137 | 2 |
| Feature (14) | Feature (4) | | 0.00129 | | | 1 |
| Feature (14) | Feature (6) | | | | 0.00115 | 1 |
| Feature (14) | Feature (7) | | | | 0.00120 | 1 |
| Feature (14) | Feature (8) | | 0.00133 | | | 1 |
| Feature (14) | Feature (12) | | | | 0.00145 | 1 |
| Feature (14) | Feature (15) | | | | 0.00117 | 1 |
| Feature (14) | Feature (18) | 0.00402 | 0.00147 | | 0.00167 | 3 |
| Feature (14) | Feature (20) | | 0.00124 | | | 1 |
| Feature (14) | Feature (22) | 0.00365 | | | 0.00114 | 2 |
| Feature (14) | Feature (27) | | 0.00130 | | 0.00132 | 2 |
| Feature (15) | Feature (14) | | | | 0.00117 | 1 |
| Feature (15) | Feature (21) | | | | 0.00117 | 1 |
| Feature (16) | Feature (6) | | | | 0.00113 | 1 |
| Feature (16) | Feature (13) | | 0.00133 | | 0.00126 | 2 |
| Feature (17) | Feature (30) | | 0.00150 | 0.00094 | | 2 |
| Feature (18) | Feature (1) | | 0.00128 | | | 1 |
| Feature (18) | Feature (3) | 0.00326 | | | 0.00144 | 2 |
| Feature (18) | Feature (5) | 0.00423 | 0.00160 | 0.00082 | | 3 |
| Feature (18) | Feature (6) | 0.00392 | 0.00148 | 0.00092 | 0.00127 | 4 |
| Feature (18) | Feature (14) | 0.00402 | 0.00147 | | 0.00167 | 3 |
| Feature (18) | Feature (19) | | | | 0.00113 | 1 |
| Feature (18) | Feature (21) | | | 0.00085 | | 1 |
| Feature (18) | Feature (22) | 0.00466 | 0.00135 | 0.00091 | | 3 |
| Feature (18) | Feature (30) | 0.00309 | 0.00121 | 0.00084 | | 3 |
| Feature (18) | Feature (31) | 0.00392 | | | | 1 |
| Feature (18) | Feature (32) | 0.00338 | | 0.00088 | | 2 |
| Feature (19) | Feature (18) | | | | 0.00113 | 1 |
| Feature (20) | Feature (5) | | | | 0.00124 | 1 |
| Feature (20) | Feature (6) | | | 0.00080 | | 1 |
| Feature (20) | Feature (14) | | 0.00124 | | | 1 |
| Feature (21) | Feature (15) | | | | 0.00117 | 1 |
| Feature (21) | Feature (18) | | | 0.00085 | | 1 |
| Feature (21) | Feature (30) | | 0.00127 | 0.00082 | | 2 |
| Feature (22) | Feature (14) | 0.00365 | | | 0.00114 | 2 |
| Feature (22) | Feature (18) | 0.00466 | 0.00135 | 0.00091 | | 3 |
| Feature (23) | Feature (30) | | 0.00188 | 0.00124 | | 2 |
| Feature (24) | Feature (6) | 0.00340 | | | | 1 |
| Feature (25) | Feature (5) | 0.00318 | | 0.00087 | | 2 |
| Feature (26) | Feature (5) | | | | 0.00112 | 1 |
| Feature (26) | Feature (6) | | | | 0.00112 | 1 |
| Feature (27) | Feature (14) | | 0.00130 | | 0.00132 | 2 |
| Feature (28) | Feature (9) | | 0.00151 | 0.00092 | | 2 |
| Feature (29) | Feature (30) | | | 0.00095 | | 1 |
| Feature (30) | Feature (1) | 0.00321 | 0.00178 | 0.00105 | | 3 |
| Feature (30) | Feature (17) | | 0.00150 | 0.00094 | | 2 |
| Feature (30) | Feature (18) | 0.00309 | 0.00121 | 0.00084 | | 3 |
| Feature (30) | Feature (21) | | 0.00127 | 0.00082 | | 2 |
| Feature (30) | Feature (23) | | 0.00188 | 0.00124 | | 2 |
| Feature (30) | Feature (29) | | | 0.00095 | | 1 |
| Feature (31) | Feature (3) | 0.00383 | | | | 1 |
| Feature (31) | Feature (5) | 0.00356 | | | | 1 |
| Feature (31) | Feature (6) | 0.00383 | | | | 1 |
| Feature (31) | Feature (18) | 0.00392 | | | | 1 |
| Feature (32) | Feature (18) | 0.00338 | | 0.00088 | | 2 |

A numerical value in each cell indicates a mutual information (MI) value of a Feature 1-Feature 2 combination. A hatched cell indicates a combination for which the MI value is not among the top 20.

In the table above, a patient who has Feature 1-Feature 2 combination is highly likely to be an ALS patient, and it is particularly effective to administer a medication containing 3-methyl-1-phenyl-2-pyrazolin-5-one to an ALS patient in an early stage of onset of the disease.

Correspondence between Features (1) to (33) and Feature, code and code type in ICD-9 code, CPT code, or HCPCS code is shown below.

| Feature | | Code | | Code Type |
|---|---|---|---|---|
| Feature (1) | Abnormality of gait | ICD-9 | 781.2 | Diagnosis |
| Feature (2) | Aldolase | CPT | 82085 | Procedure |
| Feature (3) | Antinuclear antibodies (ANA); | CPT | 86038 | Procedure |
| Feature (4) | Cervical spondylosis without myelopathy | ICD-9 | 721.0 | Diagnosis |
| Feature (5) | Creatine kinase (CK); (CPK); total | CPT | 82550 | Procedure |
| Feature (6) | Cyanocobalamin (Vitamin B-12); | CPT | 82607 | Procedure |
| Feature (7) | Degeneration of cervical intervertebral disc | ICD-9 | 722.4 | Diagnosis |
| Feature (8) | Displacement of cervical intervertebral disc | ICD-9 | 722.0 | Diagnosis |
| | without myelopathy | | | |
| Feature (9) | Dysphagia; unspecified | ICD-9 | 787.20 | Diagnosis |
| Feature (10) | Folic acid; serum | CPT | 82746 | Procedure |
| Feature (11) | Injection; gadolinium—based magnetic resonance | HCPCS | A9579 | Procedure |
| | contrast agent; not otherwise specified (nos); | | | |
| | per ml | | | |
| Feature (12) | Magnetic resonance (eg; proton) imaging; brain | CPT | 70551 | Procedure |
| | (including brain stem); without contrast material | | | |
| Feature (13) | Magnetic resonance (eg; proton) imaging; brain | CPT | 70553 | Procedure |
| | (including brain stem); without contrast | | | |
| | material; followed by contrast material(s) and | | | |
| | further sequences | | | |
| Feature (14) | Magnetic resonance (eg; proton) imaging; spinal | CPT | 72141 | Procedure |
| | canal and contents; cervical; without contrast | | | |
| | material | | | |
| Feature (15) | Magnetic resonance (eg; proton) imaging; spinal | CPT | 72148 | Procedure |
| | canal and contents; lumbar; without contrast | | | |
| | material | | | |
| Feature (16) | Magnetic resonance (eg; proton) imaging; spinal | CPT | 72156 | Procedure |
| | canal and contents; without contrast material; | | | |
| | followed by contrast material(s) and further | | | |
| | sequences; cervical | | | |
| Feature (17) | Manual therapy techniques (eg; mobilization/ | CPT | 97140 | Procedure |
| | manipulation; manual lymphatic drainage; manual | | | |
| | traction); 1 or more regions; each 15 minutes | | | |
| Feature (18) | Muscle weakness (generalized) | ICD-9 | 728.87 | Diagnosis |
| Feature (19) | Needle electromyography; 1 extremity with or | CPT | 95860 | Procedure |
| | without related paraspinal areas | | | |
| Feature (20) | Needle electromyography; 2 extremities with or | | | |
| | without related paraspinal areas | CPT | 95861 | Procedure |
| Feature (21) | Other acquired deformities of ankle and foot | ICD-9 | 736.79 | Diagnosis |
| Feature (22) | Other malaise and fatigue | ICD-9 | 780.79 | Diagnosis |
| Feature (23) | Physical therapy evaluation | CPT | 97001 | Procedure |
| Feature (24) | Protein; electrophoretic fractionation and | CPT | 84165 | Procedure |
| | quantitation; serum | | | |
| Feature (25) | Sedimentation rate; erythrocyte; automated | CPT | 85652 | Procedure |
| Feature (26) | Sedimentation rate; erythrocyte; non- | CPT | 85651 | Procedure |
| | automated | | | |
| Feature (27) | Spinal stenosis in cervical region | ICD-9 | 723.0 | Diagnosis |
| Feature (28) | Swallowing function; with | CPT | 74230 | Procedure |
| | cineradiography/videoradiography | | | |
| Feature (29) | Therapeutic procedure; 1 or more areas; each | CPT | 97112 | Procedure |
| | 15 minutes; neuromuscular reeducation of | | | |
| | movement; balance; coordination; kinesthetic | | | |
| | sense; posture; and/or proprioception for sitting | | | |
| | and/or standing activities | | | |
| Feature (30) | Therapeutic procedure; 1 or more areas; each | CPT | 97110 | Procedure |
| | 15 minutes; therapeutic exercises to develop | | | |
| | strength and endurance; range of motion and | | | |
| | flexibility | | | |
| Feature (31) | Thyroid stimulating hormone (TSH) | CPT | 84443 | Procedure |
| Feature (32) | Unspecified hereditary and idiopathic peripheral | ICD-9 | 356.9 | Diagnosis |
| | neuropathy | | | |
| Feature (33) | Immunofixation electrophoresis; serum | CPT | 86334 | Procedure |

Top 20 two-Feature combinations in 3-6, 6-9, 9-12, and 12-18 month periods prior to diagnosis plotted in Feature-to-Feature heat maps (FIGS. 1 to 4). Block representations of mutual information (MI) values of the Feature combinations are plotted at the Feature intersections on grid, with larger and darker blocks representing higher MI values.

Diagnostic labs, including antinuclear antibodies, creatine kinase, thyroid stimulating hormone, and cyanocobalamin (vitamin B-12), tend to cluster together

Muscle weakness is prominent throughout, and seems to pair with different lab tests and imaging over time.

Muscle weakness and malaise/fatigue are a strong pair of Features throughout the 18 months prior to diagnosis.

Physical therapy is an important part of two-Feature combination

TOOL #1: Patient or Physician Checklist

| | HAVE YOU EXPERIENCED |
|---|---|
| | THIS WITHIN THE |
| | PAST 3 YEARS |
| SYMPTOM OR EVENT | (CHECK ALL THAT APPLY) |
| Fatigue or Muscle Weakness | |
| Non-traumatic Joint disorder, | |
| deformities of foot or ankles | |
| Connective Tissue Disorders | |
| Skin Disorders | |
| Nervous System Disorder | |
| Any change in speech | |
| Office visit to: Physical Therapy, | |
| Neurologist, orthopedic surgeon, | |
| gastroenterologist, or otolaryngologist visits | |
| Had an imaging procedure such as | |
| EMG or MRI | |
| Prescribed any of these medications: | |
| Riluzole, Baclofen | |
| Pyridostigmine, Anticonvulsants | |

How to use TOOL #1: When symptoms or events listed in the “SYMPTOM OR EVENT” column are experienced within the last 3 years, cells on the right side corresponding to all applicable items are checked.

Interpretation: When a patient has experienced 4 or more of the 9 symptoms/events, the patient is highly likely to be an ALS patient, and it is particularly effective to administer a medication containing 3-methyl-1-phenyl-2-pyrazolin-5-one to the patient.

TOOL #2: To be Completed by Patient or Physician

| | | | OPTIONAL INFORMATION | |
|---|---|---|---|---|
| | | | TO COMPLETE | |
| | | | How many | |
| | | | months ago | |
| | | CHECK | did you FIRST | Is this |
| | | ALL | experience | symptom or |
| | | THAT | this symptom | event persistent |
| | I HAVE EXPERIENCED | APPLY | or event | (YES OR NO) |
| CATEGORY | Fatigue or Muscle Weakness | | | |
| A | Connective Tissue Disorder | | | |
| | Unusual increase in healthcare | | | |
| | resource utilization (i.e., | | | |
| | increase doctor visits, | | | |
| | procedures (MRI, EMG) new | | | |
| | diagnosis, prescriptions) | | | |
| | Nervous System Disorders | | | |
| | Skin Disorders | | | |
| CATEGORY | Prescribed medications: | | | |
| B | Riluzole, Baclofen, Pyridostigine, | | | |
| | Anticonvulsants | | | |
| | Labs: CK, Vit B 12, or ANA | | | |
| | checked or monitored | | | |
| | Foot or Ankle deformity | | | |
| | Change in Speech | | | |
| | Skin Disorder | | | |

How to use TOOL #2: When a patient has experienced symptoms or events listed in the “I HAVE EXPERIENCED” column, cells in the “CHECK ALL THAT APPLY” column corresponding to all applicable items are checked. Optionally, additional information is written in the “How many months ago did you FIRST experience this symptom or event” column and the “Is this symptom or event persistent (YES or NO)” column.

Interpretation: A patient who meets the following conditions is highly likely to be an ALS patient and it is particularly effective to administer a medication containing 3-methyl-1-phenyl-2-pyrazolin-5-one to the patient when:

The patient have experience 3 or more of the 5 symptoms/events listed in Category A, OR

The patient have experienced 2 or more symptoms or events in Category A, PLUS 3 symptoms/events in Category B

TOOL #3: To be Completed by Patient as Part of Medical History

| | Check ALL of the timeframes that you experienced this 'event' | | | | | | | | |
|---|---|---|---|---|---|---|---|---|---|
| | in each of the timeframes below, during past | | | | | | | | |
| | 0-3 | 3-6 | 6-9 | 9-12 | 12-18 | 18-24 | 24-36 | 36-48 | |
| | months | months | months | months | months | months | months | months | TOTAL |
| Fatigue or Muscle Weakness | | | | | | | | | ADD the total |
| Ankle or Foot deformity | | | | | | | | | number of |
| Connective Tissue Disorder | | | | | | | | | events across |
| Nervous System Disorder | | | | | | | | | all timeframes |
| Labs checked or monitored | | | | | | | | | |
| for CK, Vit B12, or ANA | | | | | | | | | |
| Imaging: MRI or EMG | | | | | | | | | |
| Unusually Higher Healthcare | | | | | | | | | |
| Utilization | | | | | | | | | |
| TOTAL NUMBER OF EVENTS | | | | | | | | | |

TOOL #3: Example

| | Check ALL of the timeframes that you experienced this 'event' | | | | | | | | |
|---|---|---|---|---|---|---|---|---|---|
| | in each of the timeframes below, during past | | | | | | | | |
| | 0-3 | 3-6 | 6-9 | 9-12 | 12-18 | 18-24 | 24-36 | 36-48 | |
| | months | months | months | months | months | months | months | months | TOTAL |
| Fatigue or Muscle Weakness | X | X | X | X | | x | x | x | ADD the total |
| Ankle or Foot deformity | X | X | X | X | x | X | x | | number of |
| Connective Tissue Disorder | X | X | X | X | X | | | | events across |
| Nervous System Disorder | X | X | X | x | X | | | | all timeframes |
| Labs checked or monitored | X | X | x | | | | | x | |
| for CK, Vit B12, or ANA | | | | | | | | | |
| Imaging: MRI or EMG | X | | | | | | | | |
| Unusually Higher Healthcare | X | X | | | | | | | |
| Utilization | | | | | | | | | |
| TOTAL NUMBER OF EVENTS | 7 | 6 | 5 | 4 | 3 | 2 | 2 | 2 | |

How to use TOOL #3: When a patient has experienced events listed in the leftmost column in time periods of 0-3 months, 3-6 months, 6-9 months, 9-12 months, 12-18 months, 18-24 months, 24-36 months, and 36-48 months prior to using the TOOL #3, all cells of the applicable time periods are checked.

Interpretation:

When the patient scored a 5 in any timeframe, or the patient's total is greater than 25, the patient is highly likely to be an ALS patient, and it is particularly effective to administer a medication containing 3-methyl-1-phenyl-2-pyrazolin-5-one to the patient.

TOOL #4: Algorithm that can be uploaded into an Electronic Health Record database, derivation from TOOL #3

Calculate risk potential for ALS diagnosis in the future. When the risk potential is >X, the patient is highly likely to be an ALS patient and it is particularly effective to administer a medication containing 3-methyl-1-phenyl-2-pyrazolin-5-one to the patient.

Risk Potential=(Event: Fatigue or Muscle Weakness*number of timeframes event occurred)+(Event: Ankle or Foot deformity*number of timeframes event occurred)+(Event: Connective Tissue Disorder*number of timeframes event occurred)+(Event: Nervous System Disorder*number of timeframes event occurred)+(Event: Labs checked or monitored for CK, Vit B12, or ANA*number of timeframes event occurred)+(Event: Imaging: MRI or EMG*number of timeframes event occurred)+(Event: Unusually Higher Healthcare Utilization*number of timeframes event occurred)

Assign a value of ‘1’ if the event/symptom occurred. Assign a value of ‘0’ if the event/symptom did not occur.

Add value to equation to take into account combinatorial considerations

Potential to weight the values pending the timeframe it occurred

| | Check ALL of the timeframes that you experienced this 'event' | | | | | | | |
|---|---|---|---|---|---|---|---|---|
| | in each of the timeframes below, during past | | | | | | | |
| | 0-3 | 3-6 | 6-9 | 9-12 | 12-18 | 18-24 | 24-36 | 36-48 |
| | months | months | months | months | months | months | months | months |
| Fatigue or Muscle Weakness | | | | | | | | |
| Ankle or Foot deformity | | | | | | | | |
| Connective Tissue Disorder | | | | | | | | |
| Nervous System Disorder | | | | | | | | |
| Labs checked or monitored | | | | | | | | |
| for CK, Vit B12, or ANA | | | | | | | | |
| Imaging: MRI or EMG | | | | | | | | |
| Unusually Higher Healthcare | | | | | | | | |
| Utilization | | | | | | | | |
| TOTAL NUMBER OF EVENTS | | | | | | | | |

Interpretation: When the risk potential is >X, the patient is highly likely to be an ALS patient and it is particularly effective to administer a medication containing 3-methyl-1-phenyl-2-pyrazolin-5-one to the patient.

Another exemplary model of Features including diagnoses, drugs and procedures is shown below. The Features having the same terms and phrases described above share the same definitions.

| Type | Description |
|---|---|
| Diagnosis Code | Abnormal involuntary movements |
| Diagnosis Code | Abnormality of gait |
| Diagnosis Code | Brachial neuritis or radiculitis NOS |
| Diagnosis Code | Cervical spondylosis without myelopathy |
| Diagnosis Code | Cervicalgia |
| Diagnosis Code | Degeneration of cervical intervertebral disc |
| Diagnosis Code | Displacement of cervical intervertebral disc without myelopathy |
| Diagnosis Code | Disturbance of skin sensation |
| Diagnosis Code | Dysarthria |
| Diagnosis Code | Dysphagia; unspecified |
| Diagnosis Code | Mononeuritis of unspecified site |
| Diagnosis Code | Muscle weakness (generalized) |
| Diagnosis Code | Myopathy; unspecified |
| Diagnosis Code | Other acquired deformities of ankle and foot |
| Diagnosis Code | Other malaise and fatigue |
| Diagnosis Code | Other musculoskeletal symptoms referable to limbs |
| Diagnosis Code | Other speech disturbance |
| Diagnosis Code | Pain in limb |
| Diagnosis Code | Spinal stenosis in cervical region |
| Diagnosis Code | Thoracic or lumbosacral neuritis or radiculitis; unspecified |
| Diagnosis Code | Unspecified hereditary and idiopathic peripheral neuropathy |
| Drug | Baclofen |
| Drug | Diazepam |
| Drug | Gabapentin |
| Drug | Hydrocodone & Comb. |
| Drug | Metformin & Comb. |
| Drug | Prednisone |
| Drug | Pregabalin |
| Drug | Pyridostigmine |
| Procedure Code | Creatine kinase (CK); (CPK); total |
| Procedure Code | Cyanocobalamin (Vitamin B-12); |
| Procedure Code | Magnetic resonance (eg; proton) imaging; brain (including b |
| | rain stem); without contrast material; followed by contrast material(s) |
| | and further sequences |
| Procedure Code | Magnetic resonance (eg; proton) imaging; spinal canal and |
| | contents; cervical; without contrast material |
| Procedure Code | Needle electromyography; 2 extremities with or without related paraspinal areas |
| Procedure Code | Nerve conduction, amplitude and latency/velocity study, each nerve; |
| | motor, without F-wave study |
| Procedure Code | Nerve conduction, amplitude and latency/velocity study, each nerve; |
| | motor,with F-Wave study |
| Procedure Code | Nerve conduction, amplitude and latency/velocity study, each nerve; |
| | Sensory |
| Procedure Code | Office or other outpatient visit for the evaluation and management of |
| | an established patient |
| Procedure Code | Therapeutic procedure; 1 or more areas; each 15 minutes; therapeutic |
| | exercises to develop strength and endurance; range of motion and flexibility |

“Abnormal involuntary movements” means that a patient has been diagnosed with a disease of “Abnormal involuntary movements” indicated by ICD-9 code 781.0 or has a symptom corresponding to the disease of “Abnormal involuntary movements.”

“Brachial neuritis or radiculitis NOS” means that a patient has been diagnosed with a disease of “Brachial neuritis or radiculitis NOS” indicated by ICD-9 code 723.4 or has a symptom corresponding to the disease of “Brachial neuritis or radiculitis NOS.”

“Cervicalgia” means that a patient has been diagnosed with a disease of “Cervicalgia” indicated by ICD-9 code 723.1 or has a symptom corresponding to the disease of “Cervicalgia.”

“Disturbance of skin sensation” means that a patient has been diagnosed with a disease of “Disturbance of skin sensation” indicated by ICD-9 code 782.0 or has a symptom corresponding to the disease of “Disturbance of skin sensation.”

“Dysarthria” means that a patient has been diagnosed with a disease of “Dysarthria” indicated by ICD-9 code 355.9 or has a symptom corresponding to the disease of “Dysarthria.”

“Mononeuritis of unspecified site” means that a patient has been diagnosed with a disease of “Mononeuritis of unspecified site” indicated by ICD-9 code 784.51 or has a symptom corresponding to the disease of “Mononeuritis of unspecified site.”

“Myopathy; unspecified” means that a patient has been diagnosed with a disease of “Myopathy; unspecified” indicated by ICD-9 code 359.9 or has a symptom corresponding to the disease of “Myopathy; unspecified.”

“Other musculoskeletal symptoms referable to limbs” means that a patient has been diagnosed with a disease of “Other musculoskeletal symptoms referable to limbs” indicated by ICD-9 code 729.89 or has a symptom corresponding to the disease of “Other musculoskeletal symptoms referable to limbs.”

“Other speech disturbance” means that a patient has been diagnosed with a disease of “Other speech disturbance” indicated by ICD-9 code 784.59 or has a symptom corresponding to the disease of “Other speech disturbance.”

“Pain in limb” means that a patient has been diagnosed with a disease of “Pain in limb” indicated by ICD-9 code 729.5 or has a symptom corresponding to the disease of “Pain in limb.”

“Thoracic or lumbosacral neuritis or radiculitis; unspecified” means that a patient has been diagnosed with a disease of “Thoracic or lumbosacral neuritis or radiculitis; unspecified” indicated by ICD-9 code 724.4 or has a symptom corresponding to the disease of “Thoracic or lumbosacral neuritis or radiculitis; unspecified.”

“Gabapentin” means that a patient has been prescribed a medication containing “Gabapentin” as an active ingredient.

“Hydrocodone & Comb.” means that a patient has been prescribed a medication containing “Hydrocodone or combination of Hydrocodone with other drug(s)” as an active ingredient.

“Metformin & Comb.” means that a patient has been prescribed a medication containing “Metformin or combination of Metformin with other drug(s)” as an active ingredient.

“Nerve conduction, amplitude and latency/velocity study, each nerve; motor, without F-wave study” means that a patient has received a procedure of “Nerve conduction, amplitude and latency/velocity study, each nerve; motor, without F-wave study” indicated by CPT code 95900 or an equivalent procedure.

“Nerve conduction, amplitude and latency/velocity study, each nerve; motor, with F-Wave study” means that a patient has received a procedure of “Nerve conduction, amplitude and latency/velocity study, each nerve; motor, with F-Wave study” indicated by CPT code 95903 or an equivalent procedure.

“Nerve conduction, amplitude and latency/velocity study, each nerve; Sensory” means that a patient has received a procedure of “Nerve conduction, amplitude and latency/velocity study, each nerve; Sensory” indicated by CPT code 95904 or an equivalent procedure.

“Office or other outpatient visit for the evaluation and management of an established patient” means that a patient has received a procedure of “Office or other outpatient visit for the evaluation and management of an established patient” indicated by CPT code 99212 or an equivalent procedure.

A model is applied to pre-diagnosis histories of known ALS patients and claims histories of demographically matched control patients (non-ALS control). Based on the Features selected, each patient in the group of the known ALS patients and the group of the control patients received a score representing the probability that they were an ALS patient. Varying sensitivity and specificity of the model can be attained by adjusting the probability threshold for considering a patient an ALS patient as described below.

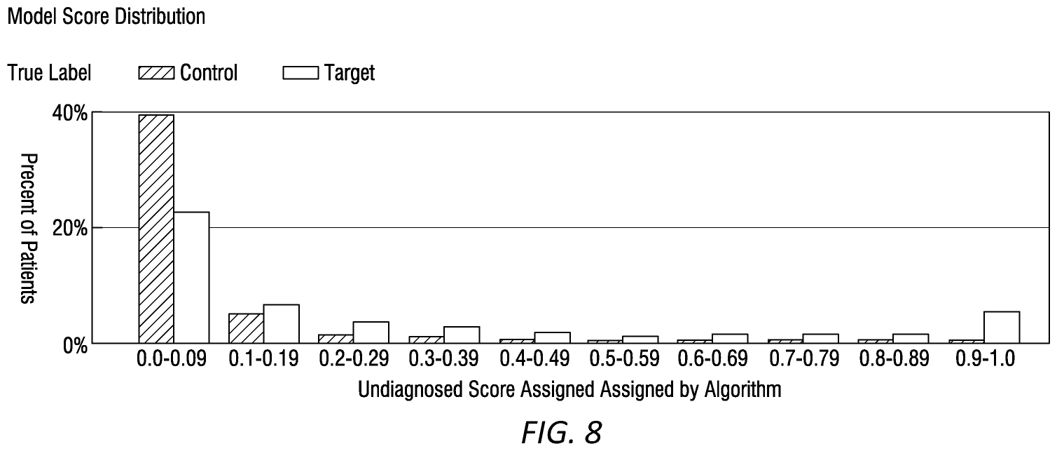

FIG. 8 illustrates score distributions of Targets (ALS patients) and Controls (control patients) of the model. In FIG. 8, the horizontal axis indicates score, and the vertical axis indicates percentage of each score for targets and controls. A higher score means a higher likelihood that a patient is a target (ALS patient).

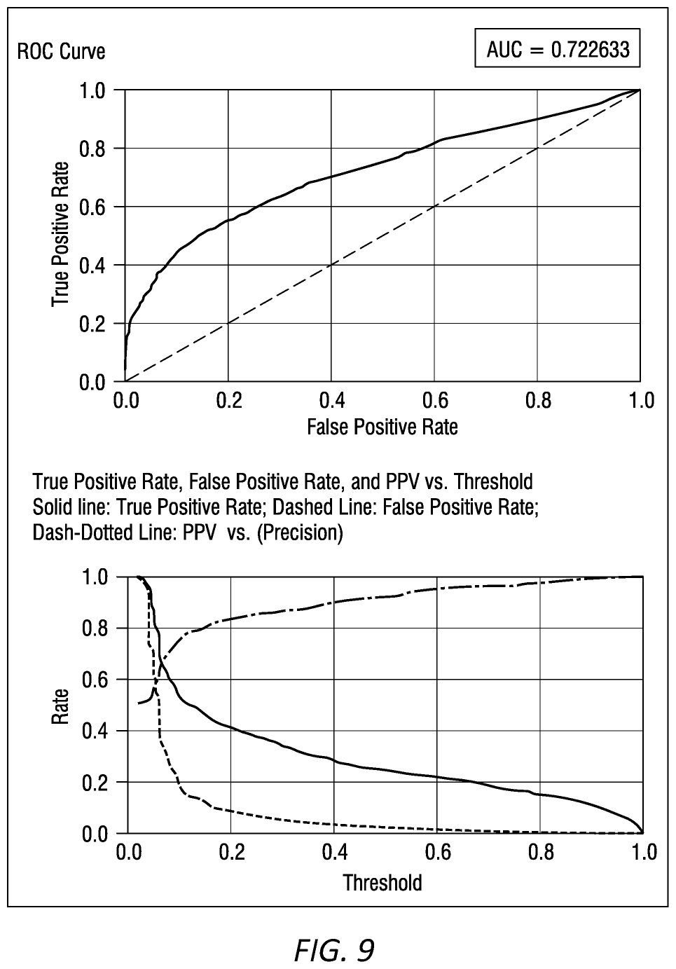

FIG. 9 illustrates ROC (Receiver Operating Characteristic) curve; True Positive Rate, False Positive Rate, and PPV vs. Threshold of the model.

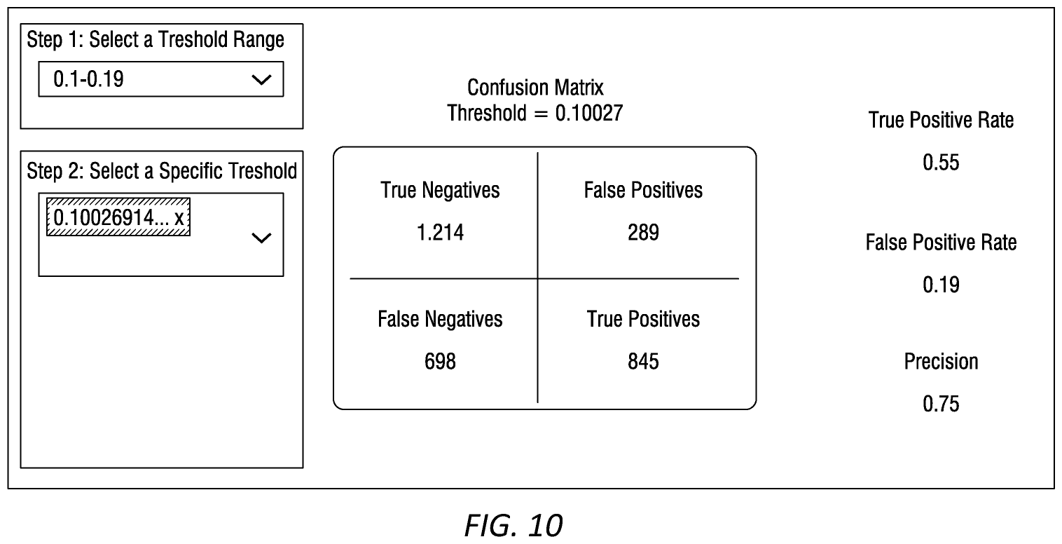

FIG. 10 illustrates a confusion matrix when the probability threshold is set to 0.1. When the probability threshold is set to 0.1, any patient with a 10% or greater probability of being an ALS patient is considered an ALS patient. This probability threshold results in good performance based on precision and true positive rate. The large number of false positives is due to a low threshold. This is a good threshold to use when true positive rate is more important than false positive rate. A lower threshold is advantageous for situations where false positives are acceptable to achieve more true positives.

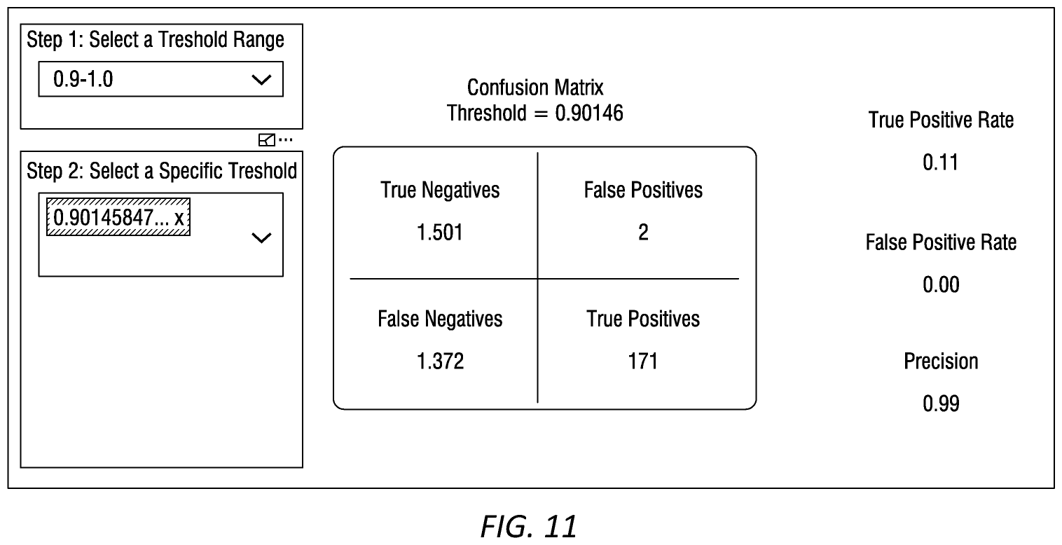

FIG. 11 illustrates a confusion matrix when the probability threshold is set to 0.9. When the probability threshold is set to 0.9, any patient with a 90% or greater probability of being an ALS patient is considered an ALS patient. This probability threshold results in good performance based on precision and false positive rate. The low number of false positives is due to a high threshold. This is a good threshold to use when false positive rate is more important than true positive rate. A higher threshold is advantageous for situations where true positives can be sacrificed to minimize false positives.

The table below shows a heat map of two-feature combinations that had the highest correlation with future ALS diagnosis compared to control patients. In the table, darker shade indicates higher relative importance of the feature combination within a given time period. Each row represents the different time periods prior to ALS diagnosis that were evaluated with MI analysis. Each column contains a combination of two features that were among the top three common differentiators (determined by MI analysis) for any individual time period prior to ALS diagnosis. In cases where a combination was among the top 3 for multiple time periods, it is listed in the earliest time period in which it appeared in the top 3, and replaced with the next highest combination in subsequent time periods in order to maintain 3 combinations per time period.

Combinations involving physical therapy and abnormality of gait begin differentiating patients as early as 48 to 60 months prior to diagnosis.

Combinations involving magnetic resonance imaging (MM) of the brain or spinal cord, muscle weakness, and electromyography (EMG) differentiate ALS patients as early as 36 to 48 months prior to diagnosis.

Combinations involving muscle weakness, other malaise or fatigue, and evaluations of serum levels of antinuclear antibodies (ANA), creatine kinase, or vitamin B-12 begin differentiating ALS patients 24 to 36 months prior to diagnosis and steadily increase as initial diagnosis approaches.

The combination of dysphagia and swallowing function becomes a significant differentiator in the year prior to diagnosis.

The combination of serum evaluation for thyroid-stimulating hormone together with muscle weakness or vitamin B-12 began to differentiate patients in the 6 months prior to diagnosis.

Echocardiography-related combinations were the top three differentiators in the 48 to 60 months prior to diagnosis, continued to differentiate through 24 months prior to diagnosis, and then were no longer included in the top 100 differentiators after 24 months.

Influenza vaccine and immunization-related combinations were among the top differentiators in the 18 to 24 months prior to diagnosis only.

Several concomitant diagnoses that were able to differentiate ALS patients were identified prior to their initial ALS diagnosis. Again, by looking at combinations of features in a patient's claims history, ALS patients were differentiated years prior to their initial ALS diagnosis. Methods according to embodiments of the present invention may be applied to identify ALS patients earlier to facilitate appropriate intervention.

| | Feature Combination | | | | | |
|---|---|---|---|---|---|---|
| | Echocardiography; | Doppler | Echocardiography; | Brain MRI; | Muscle | Brain MRI; |
| | transthoracic | echocardiography; | transthoracic | contrast material | weakness | contrast material |
| Months | Doppler | wave Doppler | Doppler | Spinal MRI; | Needle | Needle |
| Before | echocardiography | echocardiography | echocardiography; | contrast | electromyography; | electromyography; |
| Diagnosis | mapping | mapping | wave | material | 1 extremity | 1 extremity |
| 48 to 60 | 0.000597 | 0.000588 | 0.000568 | | | |
| 36 to 48 | 0.000338 | 0.000335 | 0.000388 | 0.000682 | 0.000640 | 0.000606 |
| 24 to 36 | 0.000763 | 0.000651 | 0.000717 | | 0.000747 | |
| 18 to 24 | | | | 0.000416 | 0.000564 | |
| 12 to 18 | | | | 0.001263 | 0.001132 | |
| 9 to 12 | | | | 0.000588 | 0.000622 | |
| 6 to 9 | | | | 0.001327 | 0.000932 | |
| 3 to 6 | | | | 0.002154 | | |
| 0 to 3 | | | | 0.008607 | | |

| | |
|---|---|
| 48 to 60 Months | 36 to 48 Months |

First Appearance in Top 3 Features

| | Feature Combination | | | | | |
|---|---|---|---|---|---|---|
| | Physical therapy | Radiologic | Needle | Need for | Spinal MRI; no | Need for |
| | evaluation | examination; chest | electromyography | influenza | contrast material | influenza |
| Months | Physical therapy | Needle | 1 extremity | vaccine | Needle | vaccine |
| Before | exercises; | electromyography; | Physical therapy | Influenza | electromyography; | Immunization |
| Diagnosis | strength | 2 extremities | exercises; strength | vaccine | 2 extremities | administration |
| 48 to 60 | 0.000255 | | | | | |
| 36 to 48 | 0.000366 | | 0.000333 | | | |
| 24 to 36 | 0.000900 | 0.000856 | 0.000775 | | 0.000495 | |
| 18 to 24 | 0.000793 | 0.000507 | | 0.001231 | 0.001002 | 0.000814 |
| 12 to 18 | 0.000992 | | 0.000981 | | 0.001094 | |
| 9 to 12 | 0.001239 | | 0.000561 | | | |
| 6 to 9 | 0.001883 | | | | | 0.001239 |
| 3 to 6 | 0.002882 | | | | 0.002042 | |
| 0 to 3 | | | | | | |

| | |
|---|---|
| 24 to 36 Months | 18 to 24 Months |

First Appearance in Top 3 Features

| | Feature Combination | | | | | |
|---|---|---|---|---|---|---|
| | Muscle | Cyanonobalamin | Brain MRI; no | Abnormality of gait | Abnormality | Physical therapy |
| | weakness | (Vitamin B-12) | contrast material | Physical | of gait | exercises; strength |
| Months | Spinal MRI; | Antinuclear | Spinal MRI; | therapy | Spinal MRI; | Physical therapy |
| Before | no contrast | antibodies | no contrast | exercises; | no contrast | exercises; |
| Diagnosis | material | (ANA) | material | strength | material | neuromuscular |
| 48 to 60 | | | | 0.000349 | | |
| 36 to 48 | | | | | | |
| 24 to 36 | 0.000619 | | | 0.000433 | | 0.000500 |
| 18 to 24 | 0.000597 | 0.000557 | | 0.000491 | 0.000627 | 0.000475 |
| 12 to 18 | 0.001667 | 0.001612 | 0.001447 | 0.000902 | 0.00137.3 | |
| 9 to 12 | 0.000687 | 0.001092 | 0.000585 | 0.001053 | 0.000959 | 0.000947 |
| 6 to 9 | | 0.001465 | 0.001518 | 0.000933 | 0.001775 | 0.001021 |
| 3 to 6 | 0.004021 | 0.004367 | 0.002765 | 0.003215 | 0.001939 | |
| 0 to 3 | 0.012884 | 0.013883 | 0.007380 | | | |

| | |
|---|---|
| 12 to 18 Months | 9 to 12 Months |

First Appearance in Top 3 Features

| | Feature Combination | | | | | |
|---|---|---|---|---|---|---|
| | Creatine kinase | Muscle | | | | |
| | (CK); (CPK); | weakness | | Other malaise | Creatine kinase | Muscle |
| Months | total | Creatine | Dysphagia | and fatigue | (CK); (CPK); | weakness |
| Before | Cyanocobalamin | kinase (CK); | Swallowing | Muscle | total Antinuclear | Cyanocobalamin |
| Diagnosis | (Vitamin B-12) | (CPK); total | function | weakness | antibodies (ANA) | (Vitamin B-12) |
| 48 to 60 | | | | | | |
| 36 to 48 | | | | | | |
| 24 to 36 | | 0.000704 | | 0.000389 | 0.000528 | |
| 18 to 24 | 0.000470 | 0.000791 | | 0.000388 | 0.000581 | |
| 12 to 18 | 0.001286 | 0.000948 | | 0.001057 | 0.001218 | 0.001274 |
| 9 to 12 | 0.000817 | 0.000822 | 0.000925 | 0.000912 | 0.000882 | 0.000917 |
| 6 to 9 | | 0.001716 | 0.001603 | 0.001512 | 0.001352 | 0.001352 |
| 3 to 6 | 0.004243 | 0.004229 | 0.002927 | 0.004658 | 0.004616 | 0.003925 |
| 0 to 3 | 0.017595 | 0.016528 | 0.008964 | 0.017750 | 0.014828 | 0.015396 |

| | |
|---|---|
| 6 to 9 Months | 3 to 6 Months |

First Appearance in Top 3 Features

| | Feature Combination | | |
|---|---|---|---|
| | Other malaise | Cyanonobalamin | Muscle |
| | and fatigue | (Vitamin B-12) | weakness |
| Months | Creatine kinase | Thyroid | Thyroid |
| Before | (CK); (CPK); | stimulating | stimulating |
| Diagnosis | total | hormone (TSH) | hormone (TSH) |
| 48 to 60 | | | |
| 36 to 48 | | | |
| 24 to 36 | | | |
| 18 to 24 | | | |
| 12 to 18 | | | |
| 9 to 12 | 0.000516 | | |
| 6 to 9 | 0.001478 | 0.001143 | |
| 3 to 6 | 0.002501 | 0.013782 | 0.013544 |
| 0 to 3 | 0.013905 | 0.013782 | 0.013544 |

0 to 3 Months
First Appearance in Top 3 Features

A patient having a specific Feature is highly likely to be an ALS patient and it is particularly effective to administer a medication containing 3-methyl-1-phenyl-2-pyrazolin-5-one to the patient at an early stage

According to an embodiment of the present invention, treating amyotrophic lateral sclerosis at an early stage or suppressing progress of amyotrophic lateral sclerosis at an early stage includes administering an effective amount of 3-methyl-1-phenyl-2-pyrazolin-5-one or a physiologically acceptable salt thereof to a patient who has at least two Features of identified Feature 1 to Feature 55. The identified Feature 1 to Feature 55 are selected from the following.

1. Abnormality of gait
2. Aldolase test
3. Antinuclear antibodies (ANA) test
4. Cervical spondylosis without myelopathy
5. Creatine kinase (CK):(CPK) test
6. Cyanocobalamin (Vitamin B-12) test
7. Degeneration of cervical intervertebral disc
8. Displacement of cervical intervertebral disc without myelopathy

### 9. Dysphagia

10. Folic acid; serum test
11. Serum immunofixation electrophoresis test
12. Magnetic resonance imaging test
13. Manual therapy techniques
14. Muscle weakness
15. Needle electromyography
16. Acquired deformities of ankle and foot
17. Malaise and fatigue
18. Physical therapy evaluation
19. Serum protein electrophoretic fractionation and quantitation test
20. Erythrocyte sedimentation rate test
21. Spinal stenosis in cervical region
22. Swallowing function test
23. Therapeutic procedure for neuromuscular reeducation
24. Therapeutic procedure for therapeutic exercises
25. Thyroid stimulating hormone (TSH) test
26. Unspecified hereditary and idiopathic peripheral neuropathy
27. Nervous system disorders
28. Hereditary and degenerative nervous system conditions
29. Connective tissue disease
30. Non-traumatic joint disorders
31. Multiple sclerosis

### 32. Paraplegia

### 33. Paralysis

34. Other diagnostic nervous system procedures
35. Durable Medical Equipment (DME) and supplies
36. Physical therapy

### 37. Laryngoscopy

38. Spinal puncture
39. Treatment of speech

### 40. Riluzole

### 41. Baclofen

### 42. Pyridostigmine

### 43. Anticonvulsants

### 44. Diazepam

### 45. Hydrocodone

### 46. Propoxyphene

### 47. Sympathomimetic Agents

### 48. Glycopyrrolate

### 49. Prednisone

### 50. Pregabalin

### 51. Clonazepam

### 52. Tizanidine

### 53. Levodopa or Carbidopa

### 54. Quinine

### 55. Tolterodine

In some embodiments, it is possible that a 14-day administration period and a 14-day drug holiday period are repeated, or an administration period of 10 days out of 14 days and a 14-day drug holiday period are repeated after an initial 14-day administration period followed by an initial 14-day drug holiday period. Preferably, administration periods and drug holiday periods are such that an administration period of 10 days out of 14 days and a 14-day drug holiday period are repeated after an initial 14-day administration period followed by an initial 14-day drug holiday period.

In another embodiment, drug administration can be repeated daily without providing a drug holiday period.

Preferably, symptoms caused by amyotrophic lateral sclerosis are decreased respiratory function, speech language impairment, swallowing disorder, or movement disorder of limbs.

Preferably, in a time period from 60 months before initial administration of 3-methyl-1-phenyl-2-pyrazolin-5-one or a physiologically acceptable salt thereof to the patient to the initial administration, the patient meets at least two Features among the above Feature 1 to Feature 55. A more preferred time period is from 18 months before the initial administration to the initial administration.

Further, an embodiment of the present invention includes a drug containing 3-methyl-1-phenyl-2-pyrazolin-5-one or a physiologically acceptable salt thereof as an active ingredient for treating or suppressing progress of amyotrophic lateral sclerosis. A patient receiving medication has at least two Features among identified Feature 1 to Feature 55.

The identified Feature 1 to Feature 55 are selected from the following.

1. Abnormality of gait
2. Aldolase test
3. Antinuclear antibodies (ANA) test
4. Cervical spondylosis without myelopathy
5. Creatine kinase (CK):(CPK) test
6. Cyanocobalamin (Vitamin B-12) test
7. Degeneration of cervical intervertebral disc
8. Displacement of cervical intervertebral disc without myelopathy

### 9. Dysphagia

10. Folic acid; serum test
11. Serum immunofixation electrophoresis test
12. Magnetic resonance imaging test
13. Manual therapy techniques
14. Muscle weakness
15. Needle electromyography
16. Acquired deformities of ankle and foot
17. Malaise and fatigue
18. Physical therapy evaluation
19. Serum protein electrophoretic fractionation and quantitation test
20. Erythrocyte sedimentation rate test
21. Spinal stenosis in cervical region
22. Swallowing function test
23. Therapeutic procedure for neuromuscular reeducation
24. Therapeutic procedure for therapeutic exercises
25. Thyroid stimulating hormone (TSH) test
26. Unspecified hereditary and idiopathic peripheral neuropathy
27. Nervous system disorders
28. Hereditary and degenerative nervous system conditions
29. Connective tissue disease
30. Non-traumatic joint disorders
31. Multiple sclerosis

### 32. Paraplegia

### 33. Paralysis

34. Other diagnostic nervous system procedures
35. Durable Medical Equipment (DME) and supplies
36. Physical therapy

### 37. Laryngoscopy

38. Spinal puncture
39. Treatment of speech

### 40. Riluzole

### 41. Baclofen

### 42. Pyridostigmine

### 43. Anticonvulsants

### 44. Diazepam

### 45. Hydrocodone

### 46. Propoxyphene

### 47. Sympathomimetic Agents

### 48. Glycopyrrolate

### 49. Prednisone

### 50. Pregabalin

### 51. Clonazepam

### 52. Tizanidine

### 53. Levodopa or Carbidopa

### 54. Quinine

### 55. Tolterodine

Further, an embodiment of the present invention includes 3-methyl-1-phenyl-2-pyrazolin-5-one or a physiologically acceptable salt thereof for treating or suppressing progress of amyotrophic lateral sclerosis. A patient receiving medication has at least two Features among identified Feature 1 to Feature 55.

The identified Feature 1 to Feature 55 are selected from the following.

1. Abnormality of gait

2. Aldolase test

3. Antinuclear antibodies (ANA) test

4. Cervical spondylosis without myelopathy

5. Creatine kinase (CK):(CPK) test

6. Cyanocobalamin (Vitamin B-12) test

7. Degeneration of cervical intervertebral disc

8. Displacement of cervical intervertebral disc without myelopathy

9. Dysphagia

10. Folic acid; serum test

11. Serum immunofixation electrophoresis test

12. Magnetic resonance imaging test

13. Manual therapy techniques

14. Muscle weakness

15. Needle electromyography

16. Acquired deformities of ankle and foot

17. Malaise and fatigue

18. Physical therapy evaluation

19. Serum protein electrophoretic fractionation and quantitation test

20. Erythrocyte sedimentation rate test

21. Spinal stenosis in cervical region

22. Swallowing function test

23. Therapeutic procedure for neuromuscular reeducation

24. Therapeutic procedure for therapeutic exercises

25. Thyroid stimulating hormone (TSH) test

26. Unspecified hereditary and idiopathic peripheral neuropathy

27. Nervous system disorders

28. Hereditary and degenerative nervous system conditions

29. Connective tissue disease

30. Non-traumatic joint disorders

31. Multiple sclerosis

32. Paraplegia

33. Paralysis

34. Other diagnostic nervous system procedures

35. Durable Medical Equipment (DME) and supplies

36. Physical therapy

37. Laryngoscopy

38. Spinal puncture

39. Treatment of speech

40. Riluzole

41. Baclofen

42. Pyridostigmine

43. Anticonvulsants

44. Diazepam

45. Hydrocodone

46. Propoxyphene

47. Sympathomimetic Agents

48. Glycopyrrolate

49. Prednisone

50. Pregabalin

51. Clonazepam

52. Tizanidine

53. Levodopa or Carbidopa

54. Quinine

55. Tolterodine

The embodiments of the present invention include a drug administration method and a drug useful for treating or suppressing progress of ALS or a symptom caused by ALS. Further, the drug administration method and the drug according to the embodiments of the present invention allow the drug to be administered to ALS patients at an early stage upon onset of ALS. Further, the drug administration method and the drug according to the embodiments of the present invention allow an ALS patient to be selected at an early stage upon onset of ALS and allow the drug to be administered to the patient, and allow a high therapeutic effect or a high disease progress suppression effect to be obtained.

Obviously, numerous modifications and variations of the present invention are possible in light of the above teachings. It is therefore to be understood that within the scope of the appended claims, the invention may be practiced otherwise than as specifically described herein.

1. A method for treating amyotrophic lateral sclerosis, comprising:
administering an effective amount of 3-methyl-1-phenyl-2-pyrazolin-5-one or a physiologically acceptable salt thereof to a patient who is in need thereof and meets at least two Features selected from Feature 1 to Feature 55,
wherein the Feature 1 to Feature 55 are
1. Abnormality of gait
2. Aldolase test
3. Antinuclear antibodies (ANA) test
4. Cervical spondylosis without myelopathy
5. Creatine kinase (CK):(CPK) test
6. Cyanocobalamin (Vitamin B-12) test
7. Degeneration of cervical intervertebral disc
8. Displacement of cervical intervertebral disc without myelopathy
9. Dysphagia
10. Folic acid; serum test
11. Serum immunofixation electrophoresis test
12. Magnetic resonance imaging test
13. Manual therapy techniques
14. Muscle weakness
15. Needle electromyography
16. Acquired deformities of ankle and foot
17. Malaise and fatigue
18. Physical therapy evaluation
19. Serum protein electrophoretic fractionation and quantitation test
20. Erythrocyte sedimentation rate test
21. Spinal stenosis in cervical region
22. Swallowing function test
23. Therapeutic procedure for neuromuscular reeducation
24. Therapeutic procedure for therapeutic exercises
25. Thyroid stimulating hormone (TSH) test
26. Unspecified hereditary and idiopathic peripheral neuropathy
27. Nervous system disorders
28. Hereditary and degenerative nervous system conditions
29. Connective tissue disease
30. Non-traumatic joint disorders
31. Multiple sclerosis
32. Paraplegia
33. Paralysis
34. Other diagnostic nervous system procedures
35. Durable Medical Equipment (DME) and supplies
36. Physical therapy
37. Laryngoscopy
38. Spinal puncture
39. Treatment of speech
40. Riluzole
41. Baclofen
42. Pyridostigmine
43. Anticonvulsants
44. Diazepam
45. Hydrocodone
46. Propoxyphene
47. Sympathomimetic Agents
48. Glycopyrrolate
49. Prednisone
50. Pregabalin
51. Clonazepam
52. Tizanidine
53. Levodopa or Carbidopa
54. Quinine
55. Tolterodine

2. The method according to claim 1, wherein the patient further meets at least one additional Feature selected from a skin disorder, a change in speech, an office visit to a physical therapist, a neurologist, an orthopedic surgeon, a gastroenterologist, or an otolaryngologist, an unusual increase in healthcare resource utilization including increase in doctor visit, a procedure including MRI and EMG, a new diagnosis, and a prescription, and unusually higher healthcare utilization.

3. The method according to claim 1, wherein the patient meets at least one Pair of the Features selected from Pair 1 to Pair 46, and the Pair 1 to Pair 46 are
1. Feature (1) and Feature (14)
2. Feature (1) and Feature (18)
3. Feature (1) and Feature (30)
4. Feature (2) and Feature (5)
5. Feature (3) and Feature (5)
6. Feature (3) and Feature (6)
7. Feature (3) and Feature (18)
8. Feature (3) and Feature (31)
9. Feature (4) and Feature (14)
10. Feature (5) and Feature (6)
11. Feature (5) and Feature (18)
12. Feature (5) and Feature (20)
13. Feature (5) and Feature (25)
14. Feature (5) and Feature (26)
15. Feature (5) and Feature (31)
16. Feature (6) and Feature (10)
17. Feature (6) and Feature (13)
18. Feature (6) and Feature (14)
19. Feature (6) and Feature (16)
20. Feature (6) and Feature (18)
21. Feature (6) and Feature (20)
22. Feature (6) and Feature (24)
23. Feature (6) and Feature (26)
24. Feature (6) and Feature (31)
25. Feature (7) and Feature (14)
26. Feature (8) and Feature (14)
27. Feature (9) and Feature (28)
28. Feature (11) and Feature (13)
29. Feature (12) and Feature (14)
30. Feature (13) and Feature (16)
31. Feature (14) and Feature (15)
32. Feature (14) and Feature (18)
33. Feature (14) and Feature (20)
34. Feature (14) and Feature (22)
35. Feature (14) and Feature (27)
36. Feature (15) and Feature (21)
37. Feature (17) and Feature (30)
38. Feature (18) and Feature (19)
39. Feature (18) and Feature (21)
40. Feature (18) and Feature (22)
41. Feature (18) and Feature (30)
42. Feature (18) and Feature (31)
43. Feature (18) and Feature (32)
44. Feature (21) and Feature (30)
45. Feature (23) and Feature (30)
46. Feature (29) and Feature (30)

4. The method according to claim 1, wherein the patient meets at least two Features selected from the Feature 1 to the Feature 55 during a period between a first administration of the effective amount of 3-methyl-1-phenyl-2-pyrazolin-5-one or the physiologically acceptable salt thereof to the patient and 120 months prior to the first administration.

5. The method according to claim 1, wherein the administering comprises repeating a 14-day administration period and a 14-day drug holiday period, or establishing an initial 14-day administration period and an initial 14-day drug holiday period and then repeating an administration period for 10 out of 14 days and a 14-day drug holiday period.

6. The method according to claim 1, wherein the administering comprises administering the effective amount of 3-methyl-1-phenyl-2-pyrazolin-5-one or the physiologically acceptable salt thereof to the patient every day or near every day.

7. The method according to claim 1, further comprising:
selecting the patient who meets at least two Features selected from the Feature 1 to the Feature 55 prior to a first administration of the effective amount of 3-methyl-1-phenyl-2-pyrazolin-5-one or the physiologically acceptable salt thereof to the patient.

8. A method for suppressing progress of amyotrophic lateral sclerosis, comprising:
administering an effective amount of 3-methyl-1-phenyl-2-pyrazolin-5-one or a physiologically acceptable salt thereof to a patient who is in need thereof and meets at least two Features selected from Feature 1 to Feature 55,
wherein the Feature 1 to Feature 55 are
1. Abnormality of gait
2. Aldolase test
3. Antinuclear antibodies (ANA) test
4. Cervical spondylosis without myelopathy
5. Creatine kinase (CK):(CPK) test
6. Cyanocobalamin (Vitamin B-12) test
7. Degeneration of cervical intervertebral disc
8. Displacement of cervical intervertebral disc without myelopathy
9. Dysphagia
10. Folic acid; serum test
11. Serum immunofixation electrophoresis test
12. Magnetic resonance imaging test
13. Manual therapy techniques
14. Muscle weakness
15. Needle electromyography
16. Acquired deformities of ankle and foot
17. Malaise and fatigue
18. Physical therapy evaluation
19. Serum protein electrophoretic fractionation and quantitation test
20. Erythrocyte sedimentation rate test
21. Spinal stenosis in cervical region
22. Swallowing function test
23. Therapeutic procedure for neuromuscular reeducation
24. Therapeutic procedure for therapeutic exercises
25. Thyroid stimulating hormone (TSH) test
26. Unspecified hereditary and idiopathic peripheral neuropathy
27. Nervous system disorders
28. Hereditary and degenerative nervous system conditions
29. Connective tissue disease
30. Non-traumatic joint disorders
31. Multiple sclerosis
32. Paraplegia
33. Paralysis
34. Other diagnostic nervous system procedures
35. Durable Medical Equipment (DME) and supplies
36. Physical therapy
37. Laryngoscopy
38. Spinal puncture
39. Treatment of speech
40. Riluzole
41. Baclofen
42. Pyridostigmine
43. Anticonvulsants
44. Diazepam
45. Hydrocodone
46. Propoxyphene
47. Sympathomimetic Agents
48. Glycopyrrolate
49. Prednisone
50. Pregabalin
51. Clonazepam
52. Tizanidine
53. Levodopa or Carbidopa
54. Quinine
55. Tolterodine

9. The method according to claim 8, wherein the patient further meets at least one additional Feature selected from a skin disorder, a change in speech, an office visit to a physical therapist, a neurologist, an orthopedic surgeon, a gastroenterologist, or an otolaryngologist, an unusual increase in healthcare resource utilization including increase in doctor visit, a procedure including MRI and EMG, a new diagnosis, and a prescription, and unusually higher healthcare utilization.

10. The method according to claim 8, wherein the patient meets at least one Pair of the Features selected from Pair 1 to Pair 46, and the Pair 1 to Pair 46 are
1. Feature (1) and Feature (14)
2. Feature (1) and Feature (18)
3. Feature (1) and Feature (30)
4. Feature (2) and Feature (5)
5. Feature (3) and Feature (5)
6. Feature (3) and Feature (6)
7. Feature (3) and Feature (18)
8. Feature (3) and Feature (31)
9. Feature (4) and Feature (14)
10. Feature (5) and Feature (6)
11. Feature (5) and Feature (18)
12. Feature (5) and Feature (20)
13. Feature (5) and Feature (25)
14. Feature (5) and Feature (26)
15. Feature (5) and Feature (31)
16. Feature (6) and Feature (10)
17. Feature (6) and Feature (13)
18. Feature (6) and Feature (14)
19. Feature (6) and Feature (16)
20. Feature (6) and Feature (18)
21. Feature (6) and Feature (20)
22. Feature (6) and Feature (24)
23. Feature (6) and Feature (26)
24. Feature (6) and Feature (31)
25. Feature (7) and Feature (14)
26. Feature (8) and Feature (14)
27. Feature (9) and Feature (28)
28. Feature (11) and Feature (13)
29. Feature (12) and Feature (14)
30. Feature (13) and Feature (16)
31. Feature (14) and Feature (15)
32. Feature (14) and Feature (18)
33. Feature (14) and Feature (20)
34. Feature (14) and Feature (22)
35. Feature (14) and Feature (27)
36. Feature (15) and Feature (21)
37. Feature (17) and Feature (30)
38. Feature (18) and Feature (19)
39. Feature (18) and Feature (21)
40. Feature (18) and Feature (22)
41. Feature (18) and Feature (30)
42. Feature (18) and Feature (31)
43. Feature (18) and Feature (32)
44. Feature (21) and Feature (30)
45. Feature (23) and Feature (30)
46. Feature (29) and Feature (30)

11. The method according to claim 8, wherein the patient meets at least two Features selected from the Feature 1 to the Feature 55 during a period between a first administration of the effective amount of 3-methyl-1-phenyl-2-pyrazolin-5-one or the physiologically acceptable salt thereof to the patient and 120 months prior to the first administration.

12. The method according to claim 8, wherein the administering comprises repeating a 14-day administration period and a 14-day drug holiday period, or establishing an initial 14-day administration period and an initial 14-day drug holiday period and then repeating an administration period for 10 out of 14 days and a 14-day drug holiday period.

13. The method according to claim 8, wherein the administering comprises administering the effective amount of 3-methyl-1-phenyl-2-pyrazolin-5-one or the physiologically acceptable salt thereof to the patient every day or near every day.

14. The method according to claim 8, further comprising:
selecting the patient who meets at least two Features selected from the Feature 1 to the Feature 55 prior to a first administration of the effective amount of 3-methyl-1-phenyl-2-pyrazolin-5-one or the physiologically acceptable salt thereof to the patient.

15. A method for suppressing progress of amyotrophic lateral sclerosis, comprising:
administering an effective amount of 3-methyl-1-phenyl-2-pyrazolin-5-one or a physiologically acceptable salt thereof to a patient who is in need thereof and meets at least two Features selected from Feature 1 to Feature 11,
wherein the Feature 1 to Feature 11 are
1. Malaise and fatigue, or Muscle weakness
2. Non-traumatic joint disorder or Acquired deformities of ankle and foot
3. Connective tissue disease
4. Skin disorder
5. Nervous system disorder
6. Any change in speech
7. Office visit to: Physical therapy, Neurologist, Orthopedic surgeon, Gastroenterologist, or Otolaryngologist
8. Magnetic resonance imaging test, or Needle electromyography
9. Riluzole, Baclofen, Pyridostigmine, Anticonvulsants
10. Unusual increase in healthcare resource utilization
11. Creatine kinase (CK):(CPK) test, Cyanocobalamin (Vitamin B-12) test, or Antinuclear antibodies (ANA) test.

16. The method according to claim 15, further comprising:
selecting the patient who meets at least two Features selected from the Feature 1 to the Feature 11 prior to a first administration of the effective amount of 3-methyl-1-phenyl-2-pyrazolin-5-one or the physiologically acceptable salt thereof to the patient.

17. The method according to claim 15, wherein the patient meets at least two Features selected from the Feature 1 to the Feature 11 during a period between a first administration of the effective amount of 3-methyl-1-phenyl-2-pyrazolin-5-one or the physiologically acceptable salt thereof to the patient and 120 months prior to the first administration.

18. The method according to claim 15, wherein the administering comprises repeating a 14-day administration period and a 14-day drug holiday period, or establishing an initial 14-day administration period and an initial 14-day drug holiday period and then repeating an administration period for 10 out of 14 days and a 14-day drug holiday period.

19. The method according to claim 15, wherein the administering comprises administering the effective amount of 3-methyl-1-phenyl-2-pyrazolin-5-one or the physiologically acceptable salt thereof to the patient every day or near every day.
